(19) United States
(12) Patent Application Publication
(10) Pub. No.: US 2015/0148387 A1
(43) Pub. Date: May 28, 2015

(54) METHODS OF TREATMENT, DIAGNOSIS AND MONITORING FOR METHAMPHETAMINE TOXICITY WHICH TARGET CERAMIDE METABOLIC PATHWAYS AND CELLULAR SENESCENCE

(71) Applicants: The Regents of the University of California, Oakland, CA (US); Fondazione Istituto Italiano di Tecnologia, Genova (IT)

(72) Inventors: Daniele Piomelli, Irvine, CA (US); Giuseppe Astarita, Laguna Beach, CA (US); Agnesa Avanesian, Irvine, CA (US); Andrea Cavalli, San Lazzaro di Savena (BO) (IT)

(21) Appl. No.: 14/502,990

(22) Filed: Sep. 30, 2014

Related U.S. Application Data
(63) Continuation of application No. PCT/US2013/034834, filed on Apr. 1, 2013.
(60) Provisional application No. 61/618,361, filed on Mar. 30, 2012.
(60) Provisional application No. 61/806,335, filed on Mar. 28, 2013.

(51) Int. Cl. A61K 31/4152; A61K 31/137

(52) U.S. Cl. CPC A61K 31/4152; A61K 31/137
USPC 514/380

(57) ABSTRACT

Methamphetamine is a highly addictive psychostimulant that causes profound damage to the brain and other body organs. Post mortem studies of human tissues have linked the use of this drug to diseases associated with aging, such as coronary atherosclerosis, but the molecular mechanism underlying these findings remains unknown. We report now that methamphetamine accelerates cellular senescence in vitro and activates transcription of genes involved in cell-cycle control and inflammation in vivo by stimulating production of the sphingolipid messenger ceramide. This pathogenic cascade is triggered by reactive oxygen species, generated through methamphetamine metabolism via cytochrome P₄₅₀-2D6, which recruit nuclear factor (NF)-KB to induce expression of enzymes in the de novo pathway of ceramide biosynthesis. Inhibitors of ceramide formation prevent methamphetamine-induced senescence and attenuate systemic inflammation and health deterioration in rats self-administering the drug. The results support therapeutic approaches to reduce the adverse consequences of methamphetamine abuse and improve effectiveness of treatments.

### CROSS-REFERENCES TO RELATED APPLICATIONS

This application claims priority benefit of U.S. Provisional Patent Application Ser. No. 61/806,335, filed on Mar. 28, 2013 and U.S. Provisional Patent Application Ser. No. 61/618,361 filed on Mar. 30, 2012, which are incorporated by reference herein in their entireties for all purposes.

### STATEMENT AS TO RIGHTS TO INVENTIONS MADE UNDER FEDERALLY SPONSORED RESEARCH AND DEVELOPMENT

This invention was made with Government support under Grant No. DA028902, awarded by the National Institutes of Health. The Government has certain rights in this invention.

### REFERENCE TO A “SEQUENCE LISTING,” A TABLE, OR A COMPUTER PROGRAM LISTING APPENDIX SUBMITTED ON A COMPACT DISK

Not Applicable

### BACKGROUND OF THE INVENTION

Methamphetamine addicts show profound signs of accelerated aging, but the mechanism underlying this phenomenon is still unknown. The use of the street drug, methamphetamine, has been shown to promote mtDNA deletions and increase oxidative damage, two parameters which have been shown to have an inverse relationship with lifespan in mammals [1-3]. Additional signs of accelerated aging in methamphetamine users include premature myocardial infarctions, atherosclerosis, cardiomyopathy and decline in kidney function [4]. Most practitioners note a much older appearance in patients using methamphetamine after only a few years of use, thus we set out to identify molecular mechanisms that may be involved in methamphetamine induced accelerate aging.

To treat intoxication by amphetamines, the following drugs are currently used: benzodiazepines, dopamine-receptor antagonists (e.g., roperidol or haloperidol, butyrophenones) and antipsychotics (e.g., olanzapine and risperidone). However, these drugs act mainly on the central nervous system and do not target peripheral tissues that undergo systemic inflammatory syndrome, senescence and multi-organ failure as consequence of amphetamine toxicity. Ceramide has long been implicated as a molecular modulator of aging and longevity [6]. The first evidence of this was seen with the LAG1 mutants and further supported by finding of the role of ceramide in inducing cellular senescence [10]. Here, we report that methamphetamine acts via ceramide to induce cellular senescence and increased chronological aging and that these adverse effects of amphetamines can be treated by manipulation of ceramide metabolism and also apoptosis. Accordingly, this invention provides new methods for treating and diagnosing methamphetamine-induced systemic inflammatory syndrome, senescence and organ failure.

### BRIEF SUMMARY OF THE INVENTION

The invention presented herein provides a method to decrease the toxic effects and morbidity (e.g., accelerated senescence), and prevent complications (e.g., organ failure, multi-organ failure, death) that arise from abuse of amphetamine-type drugs. In this aspect, the present invention discloses the use of inhibitors of de-novo ceramide biosynthesis (e.g., L-cycloserine) or ceramide actions (e.g. thalidomide) as agents for alleviating the systemic toxicity associated with the use of amphetamines/amphetamine-type drugs. Oral, topical, intramuscular or intravenous administration of such inhibitors attenuates injuries induced by amphetamines, reducing their toxic effects (e.g., accelerated senescence) and complications (e.g., multi-organ failure).

Accordingly, in this first aspect, the invention provides a method of treating an amphetamine-type drug-induced toxicity, said method comprising administering to the subject in need thereof an effective amount of modulator of ceramide metabolism or apoptosis which counters an effect of the drug on ceramide levels or ceramide metabolism or apoptosis. In some embodiments, the treating reduces, prevents or delays the development of an amphetamine-type drug toxicity selected from induced senescence or organ failure in the subject. In still further embodiments of any of the above, the toxicity is mediated by an amphetamine-type drug-induced increased ceramide signaling in apoptosis. The amphetamine-type drug may also be administered to the subject before or after the modulator and at a therapeutically effective time with respect to administering the amphetamine-type drug to the subject. The modulator can be administered for instance from about 15 minutes to about 24 hours before administering the amphetamine-type drug, about 2 to 4 hours before administering the amphetamine-type drug, or at about the same time the amphetamine-type drug. The modulator can be administered well after the amphetamine-type drug and for as long as its adverse effects on health and/or ceramide levels would linger. The modulator can be a ceramide synthesis inhibitor and/or an antisense nucleic acid, a ribozyme, a triplex-forming oligonucleotide, a siRNA, a probe, a primer, an antibody or a combination thereof. In some embodiments, an agent that inhibits ceramide biosynthesis targets at least one ceramide-biosynthetic enzyme selected from the group consisting of a sphingomyelinase, serine palmitoyltransferase, 3-ketosphinganine reductase, ceramide synthase, dihydroceramide desaturase, and combinations thereof. In some further embodiments of the above, the modulator can be FB1, D609, myriocin, cyclosporine, thalidomide, lenalidomide and combinations thereof. In still other embodiments, the modulator is adalimumab, golimumab, infliximab, natalizumab, etanercept, Certolizumab pegol, or Pegsunercept. In other embodiments of any of the above, toxicity is atherosclerosis, cardiomyopathy, cardiac infarction, cardiac insufficiency, or a decline in kidney function. In yet further embodiments of any of the above, the amphetamine-type drug is amphetamine, dextroamphetamine, ephedrine, pseudoephedrine, methamphetamine or a pharmaceutically acceptable salts thereof.

Further, the invention provides a method for monitoring with ease, low-invasiveness and low-cost peripheral biomarkers of amphetamine-type drug toxicity (i.e., ceramides), which could find potential applications in the following areas: (1) prophylactic and diagnostic screening in a large population of subjects; (2) leading to a more accurate diagnostic tool, especially if used in combination with other clinical parameters; (3) assessing drug response in asymptomatic patients; (4) serving as a secondary outcome measure in clinical trials of symptomatic patients, and (5) deciding if further development of a treatment should be stopped if not likely to be effective; (6) screening compounds for activity in modulating amphetamine toxicity. Further, we disclose a new set of lipid biomarkers ceramide species including, but not limited to, Cer(16:0), Cer(16:1), Cer(18:0), Cer(20:0), Cer(20:1), Cer(24:1); and dihydroceramide species including, but not limited to, DHCer(16:0), DHCer(16:1), DHCer(18:0), DHCer(20:0), DHCer(20:1), DH Cer(24:1) which can be monitored to diagnose and monitor the toxicity induced by amphetamines.

### BRIEF DESCRIPTION OF THE DRAWINGS

FIG. 1. Lipidome-wide profiles in various tissues of rats self-administering D-meth. (a) Heat-map showing changes in the levels of lipid classes (rows) in rats exposed to methamphetamine for 8 days, compared to control rats receiving saline injections. (b) Heat-map showing changes in the levels of various ceramide species (rows) in rats self-administering methamphetamine relative to control rats; columns show data from individual animals. (c-g) Levels of (c) ceramide, (d) dihydroceramide (DHC), (e) sphingomyelin (SM), (f) dihydrosphingomyelin (DHSM), and (g) mRNAs encoding for enzymes of de novo ceramide biosynthesis in skeletal muscle (vastus lateralis); control (C), open bars; rats self-administering methamphetamine (M), filled bars. DH-, dihydro-; P-, phosphatidyl-; serine palmitoyl transferase, SPT; ceramide synthase, CerS. (h) Dose-dependent effects of involuntary acute administration of methamphetamine on ceramide (d18:1/16:0) levels in rat skeletal muscle.

FIG. 2. Effects of methamphetamine on de novo ceramide biosynthesis in primary mouse embryonic fibroblasts (MEF). (a) Concentration dependence of the effect of methamphetamine on ceramide (d18:1/16:0) levels. (b) Effects of vehicle, d-methamphetamine, 1-methamphetamine, d-amphetamine, and cocaine on ceramide (d18:1/16:0) levels. (c) mRNA levels of the genes involved de novo ceramide biosynthesis. (d-f) Lipid analyses of isolated mitochondria from MEF treated with methamphetamine (1 mM). (d) short chain ceramide (d18:1/16:0); (e) long chain ceramide (d18:1/24:0; (f) long chain ceramide d18:1/24:1. (g-i) Effects of ceramide synthesis inhibitors on methamphetamine induced synthesis of ceramides n primary MEF. (g) 50 μM Fumonisin B1 (FB1), a potent inhibitor of CerS; (h) 30 μM L-cycloserine (L-CS) an inhibitor of SPT; (1) 10 μM myrocin.

FIG. 3. Effects of methamphetamine on cell senescence in MEF cells. MEFs were treated with methamphetamine (1 mM) for 48 h and senescence associated β-galactosidase (SA-β-gal) levels were measured. (a) Effects of methamphetamine exposure on the number of SA-β-gal positive cells from passage 1 to 5 compared to vehicle-treated cells. (b) Morphological changes typical of senescent phenotype. (c, d) Effects of methamphetamine treatment on measures of replicative capacity. (c) DNA synthesis was evaluated using [³H]-thymidine binding. (d) number of population doublings compared to control cells. (e) Ceramide levels over passages 1 to 5; (f) CerS5 levels over passages 1 to 5. (g, h) Effects of blockage of ceramide synthesis and/or ceramide substitution on methamphetamine induced senescence. MEFs were treated with methamphetamine (1 mM) in the presence of structurally distinct inhibitors of de novo ceramide biosynthesis. Effect of treatments with L-CS (30 μM) or FB1 (50 μM) and/or ceramide analog C8 on (g) SA-β-gal expression and (h) the percent of senescent cells.

FIG. 4. (a-d). Effects of methamphetamine on the transcription of inflammatory cytokines and its antagonism by L-CS. (a). IL-6; (b). TNF-alpha; (c) cyclin dependent kinases p21 and (d) p53. (e). Effects of methamphetamine on NF-KB activation. MEFs treated with methamphetamine (1 mM) were harvested 24 hrs later and subjected to chromatin immunoprecipitation assays to assess recruitment of NF-kB subunit p65 to the TNF-α promoter. (f). Effects of methamphetamine and TNF-α treatment on ceramide (d18:1/16:0) levels. (g-i). Effects of three NF-κB inhibitors, (g). thalidomide; (b) 5′-aminosalicyclic acid and (i) JSH-23 on methamphetamine induced de novo ceramide (d18:1/16:0) biosynthesis.

FIG. 5. (a-h) Effects of methamphetamine self-administration or acute d-methamphetamine treatment on the transcription of age-related genes. Self-administration: (a) TNF-α, (b) IL-6, (c) p21, and (d) p53. Acute d-methamphetamine treatment (e) TNF-α, (f) IL-6, (g) p21, and (h) p53. (i-k) Effects of blocking ceramide biosynthesis in mice administered methamphetamine alone or methamphetamine in combination with the SPT inhibitor L-CS on (i) ceramide content; (j) the expression of IL-6 mRNA; and (k) the expression of p21 mRNA. (l-o) Effects of L-cycloserine on (l) ceramide levels; (m) IL-6 expression; (n) p21 expression; and (o) body weight in mice self-administering methamphetamine for 8 days with or without a co-treatment with L-cycloserine starting on Day 4 of the self-administration.

FIG. 6. Methamphetamine self-administration in rats closely mimics the voluntary component of human drug exposure, and is characterized by high rates of drug intake (FIG. 1). (a) Intake of drug (mg/kg) per session. (b) Number of active hole responses vs. non-active hole responses.

FIG. 7. Effect of L-CS (L-cycloserine) on methamphetamine induced increases in ceramide in isolated mitochondria from primary MEF. (a) Cer (d18:1/16:0); (b) Cer (d18:1/24:0); (c) Cer (d18:1/24:1). Values are in pmol/mg of protein.

FIG. 8. Effect of L-CS (L-cycloserine) on methamphetamine self-administration. (a) Intake of methamphetamine (mg/kg) per session. (b) Number of active hole responses vs. non-active hole responses.

FIG. 9. Role of cytochrome P450 (CYP) in methamphetamine-induced ceramide production. (a,b) Effects of CYP inhibitor clotrimazole (CLO) on (a) cell-associated methamphetamine content and (b) ceramide levels. Primary MEF cultures were treated with methamphetamine (M, 1 mM) for 24 h and rinsed before extraction and quantification of methamphetamine by LC/MS. (e) Effects of CYP inhibitors on ceramide levels: SKF-525A (SKF, 10 μM), cimetidine (CIM, 10 μM), quinidine (QUI, 10 μM) and HET-0016 (HET, 10 μM). (d-e) Time-course of the effects of (d) methamphetamine (mM) and (e) 4-hydroxy-D-methamphetamine (4-OH, 1 mM) on ROS production. (f-g) Effects of (f) clotrimazole and (g) SKF-525A, cimetidine, quinidine and HET-0016 on ROS production. Values are expressed as mean±s.e.m. of three separate experiments, each performed in triplicate. ANOVA followed by Bonferroni post hoc test: ***P<0.001 vs vehicle; ^{s}P<0.05, ^{SSS}P<0.001 vs methamphetamine.

FIG. 10. Time-course of hydrogen peroxideproduction in primary MEF cultures treated with D-methamphetamine or L-methamphetamine (each at 1 mM). ***P<0.01, two-tailed Student's t test.

FIG. 11. Effects of methamphetamine self-administration, L-cycloserine (L-CS) treatment or combination of methamphetamine plus L-CS on food intake. *P<0.05, **P<0.01, ***P<0.001, ANOVA followed by Bonferroni post hoc test.

### DETAILED DESCRIPTION OF THE INVENTION

In the present study, we used an unbiased lipidomic approach to identify the mechanism behind amphetamine-type drugs (e.g., methamphetamine)-induced aging and senescence. Our results have implicated that alterations in ceramide biosynthesis are responsible for the evolution of many pathologies attributed to amphetamine-type drug use (e.g., methamphetamine use) and provide the rational for development of novel therapeutic interventions. More specifically we found that alterations in de novo ceramide metabolism, caused by methamphetamine, lead to drug-induced senescence. Although, the aging consequences of methamphetamine addiction in people were phenotypically obvious, not much was known about the molecular mechanisms responsible for this process. We have shown that methamphetamine can accelerate aging in vivo and in vitro by increasing the rate at which cells senescence and by inducing a state of chronic systemic inflammation two robust markers of aging. Of even more significance is the fact that the induction of senescence and inflammation induced peripherally by methamphetamine use is dependent on increased cellular ceramide contents and that by blocking the induction of ceramide biosynthesis with L-CS we are able to ameliorate the premature aging consequences of amphetamine-type drug use (e.g., methamphetamine use).

Accordingly, this invention provides for the use of modulators of ceramide biosynthesis or ceramide action, to ameliorate the toxicity and systemic inflammation associated with abuse (acute or chronic) or other uses (acute or chronic) of amphetamine-type drugs. Pharmacological modulation of drug-induced toxicity and systemic inflammation is a highly desirable therapeutic intervention. Furthermore, we describe a set of biological measurements from easily accessible human tissues (e.g. blood), which are strong indicators or predictors of systemic toxicity associated with abuse of amphetamine-type drugs. Early detection of amphetamine-type drug-induced toxicity is highly desirable to monitor the progression of the amphetamine-type drug toxicity, to assess responses to drug treatments and improve therapeutic intervention.

We identified select lipid species that are altered in the dorsal striatum of male Sprague-Dawley rats treated with a dose of methamphetamine (2×10 mg/kg; intraperitoneal injection), which produces neurotoxicity in rats. These alterations reveal previously unknown and potentially important effects of methamphetamine on the rat brain lipid interactome. Most notably, we found that methamphetamine administration is followed by a marked increase in the striatal levels of various ceramide species, which are known to be involved in cell aging and apoptotic cell death. RT-PCR analyses showed that the expression of mRNA transcripts encoding for ceramide synthases isoforms were markedly and selectively elevated in the dorsal striatum of methamphetamine-treated rats, compared to saline-treated controls. These results indicate that methamphetamine enhances de novo ceramide biosynthesis in the dorsal striatum, a brain region that is highly sensitive to methamphetamine toxicity. Although drug addiction is conceptualized as a chronic disease of the brain, exposure to drugs can also affect a variety of extra-neural tissues. In particular, amphetamine-derived stimulant drugs such as methamphetamine are known to induce disruptive effects on mitochondrial function, which are also evident in the liver. These data suggest that peripheral tissues might provide a source of biomarkers for exposure to amphetamines. No information is currently available, however, about the possible association of amphetamine use with peripheral lipid dysfunction. Encouraged by the results obtained in the brain, we conducted lipidomic analyses of liver tissue from rats exposed to methamphetamine (2×10 mg/kg; intraperitoneal injection). The analyses revealed marked increases in ceramide levels in various peripheral tissues including plasma, skeletal muscle, heart, liver and skin. Our choice of tissues is based on two criteria: (i) existence of metabolic links with lipid pools of the brain; and (ii) ease of access for potential biomarker collection. Thus, we found that exposure to a toxic dose of methamphetamine alters lipid profiles not only in the brain, but also in peripheral tissues that are vulnerable to the toxic effects of this drug. Notably, low doses (i.e., non-toxic) of methamphetamine did not induce any changes in ceramide species. Our data suggest that peripheral tissues provide a source of biomarkers for abuse of amphetamines and consequent toxicity. Additionally, our data revealed that the inhibition of the de-novo ceramide biosynthesis (e.g.; by L-cycloserine) or ceramide actions (e.g., thalidomide) is able to block the toxic effects induced by amphetamine. In particular, these drugs prevent amphetamine-induced inflammation and senescence (measured by beta-galactosidase assay, crystal violet morphology, and gene expression of pro-inflammatory and pro-senescence genes). This evidence corroborates the use of the de-novo ceramide biosynthesis (e.g., by L-cycloserine) or actions (e.g., thalidomide) to decrease amphetamine toxicity.

Oral, topical, intramuscular or intravenous administration of inhibitors of de-novo ceramide biosynthesis or inhibitors of ceramide action attenuates injuries induced by amphetamine-type drugs, reducing the toxic effects and morbidity and prevent complications (e.g., multi-organ failure).

### Amphetamine-Type Drugs

Amphetamine-type drugs act as central nervous system stimulants and have many therapeutic uses as wells as much potential for abuse. These drugs generally possess an phenyethylamine core. Amphetamine-type drugs according to the invention include amphetamine, its dextro and levo racemates, dextroamphetamine, ephedrine, pseudoephedrine, methamphetamine, methylphenidate and their salts (e.g., amphetamine sulphate, dextroamphetamine and methamphetamine). These drugs may also be co-formulated in the pharmaceutical compositions according to the invention.

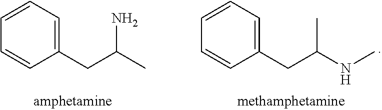

### Amphetamine-Type Drug Induced Toxicity

Amphetamine-type drug induced toxicities include adverse effects in the Central and Peripheral Nervous Systems, and non-nervous system organs such as the heart, kidneys, circulatory system, skin, pancreas, and lungs. Adverse effects include, but are not limited to, early cell death and loss of function for the affected organs. Adverse effects include, but are not limited to, cardiac insufficiency, cardiomyopathy, heart failure, atherosclerosis, reduced kidney function or kidney failure, inflammation, and type 2 diabetes. Adverse effects are dose-related, increase with increasing dose, and can result from acute and/or chronic administration of the amphetamine drug(s).

### Modulators/Agents/Compounds for Use in Treating Amphetamine-Type Drug Induced Toxicity According to the Invention.

Ceramide acts as a second messenger in the apoptotic cascade. Diverse cytokine receptors and environmental stresses utilize ceramide to signal activation of apoptosis. (see, Haimovitz-Friedman et al., British Medical Bulletin 53(3):539-553 (1997); and see also, Bikman et al., Journal of Clinical Investigation 121(11):4222-4230 (2011), each of which is incorporated by reference with respect to the modulators which reduce ceramide levels, functional elements of the apoptotic cascade which comprise a ceramide moiety, modulators which reduce the apoptotic cascade, and/or favor anabolism over catabolism as disclosed therein). Accordingly, reducing ceramide levels and/or other elements of the apoptotic cascade is contemplated to treat amphetamine-type drug (e.g., methamphetamine)-induced senescence and aging.

Modulators of apoptosis for use according to the invention further include the TNF alpha inhibitors adalimumab, golimumab, infliximab, natalizumab, etanercept, Certolizumab pegol, and Pegsunercept.

Modulators of ceramide metabolism for use according to the present invention also include those agents disclosed in U.S. Patent pplication No. 20030096022, published May 22, 2003, corresponding to U.S. patent application Ser. No. 10/029,372 filed on Dec. 21, 2001 and incorporated herein by reference in its entirety with respect to such agents and their use in reducing ceramide levels or apoptosis. These include inhibitors of reactions that yield metabolic precursors of ceramide, which is a metabolic precursor of SPH and S-1-P. Enzymes that catalyze such reactions include but are not limited to serine palmitoyl transferase (SPT) which catalyzes the production of 3-ketosphinganine, a precursor in ceramide synthesis (see, Methods in Enzymology, 311:1-9, 1999). Inhibitors of serine palmitoyl transferase include but are not limited to viridiofungins (e.g., Australifungin, Viridiofungins, Rustmicin, and Khafrefungin) (see, Mandala et al., J. Antibiot. (Tokyo) 50:339-343, 1997; and Mandala et al., Methods in Enzymology, 311:335-348, 1999), lipoxamycin (Mandala et al., J. Antibiot. (Tokyo) 47:376-379, 1994), and sphingofungins E and F (Horn et al., J. Antibiot. (Tokyo) 45:1692-1696, 1992). Other SPT inhibitors are disclosed by Hanada et al., Biochemical Pharmacology, 59:1211-1216, 2000; Zweerink et al., The Journal of Biological Chemistry, 267:25032-25038, 1992; and Riley ct al., Methods in Enzymology, 311:348-361, 1999). 3-Ketosphiganine Reductase catalyzes the production of sphinganine (dihydrosphingosine), a precursor in ceramide synthesis. See Beeler et al., The Journal of Biological Chemistry, 273:30688-30694, 1998. Dihydroceramide synthase catalyzes the acetylation of dihydrosphingosine to produce dihydroceramide, a direct precursor of ceramide. Inhibitors of ceramide synthase include, but are not limited to, Fumonisin B1 (a fungal toxin) (Merrill et al., J. Lipid Res. 26:215-234A, 1993; Wang et al., Adv. Lipid Res. 26:215-234, 1993; Tsunoda et al., J. Biochem. Mol. Toxicol. 12:281-289, 1998); derivatives of fumonisin (Humpf et al., J. Biol. Chem. 273:19060-19064, 1998); alternaria toxins (Mandala et al., J. Antibiot. 48:349-356, 1995); viridiofungins (Merrill et al., J. Lipid Res. 26:215-234A, 1993); astralifungins (Mandala et al., J. Antibiot. 48:349-356, 1995; Furneisen et al., Biochim. Biophys. Acta. 1484:71-82, 2000); and D-erythro-N-myristoyl 2-amino-1-phenylpropanol (Hunnan, Science 274:1855-1859, 1996). Agents which stimulate the destruction of metabolic precursors of ceramide are also contemplated for use according to the present invention. Enzymes that catalyze such reactions include but are not limited, sphingomyelin deacylase which catalyzes the production of sphingoylphosphorylcholine from sphingomyelin.

Additional modulators of ceramide levels for use according to the present invention are disclosed in U.S. Patent Publication No. 20050182020, published on Aug. 18, 2005, corresponding to U.S. patent application Ser. No. 10/712,684, filed on Nov. 14, 2003 and which is incorporated herein by reference with respect to such modulators (a) myriocin; (b) cycloserine; (c) Fumonisin B1; (d) PPMP; (e) compound D609; (f) methylthiodihydroceramide; (g) propanolol; and (h) resvaratrol. Additional deoxynojirimycin derivative modulators for use according to the invention are disclosed in U.S. Patent Publication No. 20070135487, published on Jun. 14, 2007, and corresponding to U.S. patent application Ser. No. 10/595,584, filed on Oct. 29, 2004, and incorporated herein by reference with respect to such derivatives disclosed therein and their methods of administration. Suitable deoxynojirimycin derivatives for use according to the present invention are disclosed in EP 947, EP 193770, U.S. Pat. No. 4,940,705, EP 481950, WO 95/22975, WO 00/33843, WO 01/07078 which are each hereby incorporated by reference with respect to such subject matter.

As taught in U.S. Patent Publication No. 20080241121, published Oct. 2, 2008, and corresponding to U.S. patent application Ser. No. 11/695,519, filed Apr. 2, 2007, and incorporated by reference in its entirety with respect to the ceramide modulating agents disclosed therein, a number of other agents can be used to reduce ceramide levels. By way of example inhibitors of SPT include, but are not limited to, the sphingo-fungins, lipoxamycin, myriocin, L-cycloserine and β-chloro-L-alanine, as well as the class of Viridiofungins. Ceramide synthase acylates the amino group of sphingosine, sphinganine and other sphingoid bases using acyl CoA esters. Inhibitors of this enzyme include, for example, the Fumonisins, the related AAL-toxin, and australifungins. The Fumonisins family of inhibitors are produced by Fusarium verticillioides and includes Fumonisin B1 (FB1). The N-acylated forms of FB1 are potent ceramide synthase inhibitors as the O-deacylated form is less potent. Of the N-acylated forms of FB1, the erythro-, threo-2-amino-3-hydroxy-, and stereoisomers of 2-amino-3,5-dihydroxyoctadecanes are contemplated. Australifungins from the organism Sporomiella australlis are also contemplated for use according to the invention as they inhibit ceramide synthase as well. Contemplated inhibitors of dihydroceramide desaturase include but are not limited to the cyclopropene-containing sphingolipid GT11, as well as a-ketoamide (GT85, GT98, GT99), urea (GT55) and thiourea (GT77) analogs of this molecule. Sphingomyelin pathway inhibitors are also contemplated for use according to the invention. Sphingomyelin is hydrolyzed by sphingomyelinase to yield phosphorylcholine and ceramide. The physiological inhibitors of sphingomyelinase are also contemplated for use according to the invention and include L-alpha-phosphatidyl-D-myo-inositol-3,5-bisphosphate, L-alpha-phosphatidyl-D-myo-inositol-3,4,5-triphosphate. Ceramide-1-phosphate and sphingosine-1-phosphate are also so contemplated. Glutathione is another agent for use according to the methods of the invention. Compounds, which are structurally unrelated to sphingomyelin, but function as sphingomyelinase inhibitors can also be used according to the invention. These compounds include desipramine, imipramine, SR33557, (3-carbazol-9-yl-propyl)-[2-(3,4-dimethoxy-phenyl)-ethyl)-methyl-amine (NB6), Hexanoic acid (2-cyclo-pent-1-enyl-2-hydroxy-1-hydroxy-methyl-ethyl)-amide (NB12) C11AG and GW4869. Compound SR33557 is a specific acid sphingomyelinase inhibitor. Other inhibitors for use according to the invention which are derived from natural sources include Scyphostatin, Macquarimicin A, and Alutenusin, Chlorogentisylquinone, and Manumycin A, and alpha-Mangostin. Scyphostatin analogs can also be used according to the invention (e.g., spiroepoxide 1, Scyphostatin and Manumycin A sphingolactones). Sphingomyelin analogs with inhibitory proprieties are also contemplated for use according to the invention (e.g., 3-O-methylsphingomyelin, and 3-O-ethylsphingomyelin).

The following compounds which have been shown to reduce ceramide by inhibition can also be used according to the invention: [3 (10,11-Dihydro-dibenzo[b,f]azepin-5-yl)-N-propyl]-[2 (3,4-dimethoxyphenyl)-ethyl]methylamin, [3 (10,11-Dihydro-dibenzo[b,f]azepin-5-yl)-N-propyl]-[2 (4-methoxyphenyl)-ethyl]methylamin, [2 (3,4-Dimethoxyphenyl)-ethyl]-[3 (2-chlorphenothiazin-10-yl)-N-propyl]-methylamin, [2 (4-Methoxyphenyl)-ethyl]-[3 (2-chlorphenothiazin-10-yl)-N-propyl]-methylamin, [3 (Carbazol-9-yl)-N-propyl]-[2 (3,4-dimethoxyphenyl)-ethyl]methylamin, [3 (Carbazol-9-yl)-N-propyl]-[2 (4-methoxyphenyl)-ethyl]methylamin, [2 (3,4-Dimethoxyphenyl)-ethyl]-[2 (phenothiazin-10-yl)-N-ethyl]-methylamin, [2 (4-Methoxyphenyl)-ethyl]-[2 (phenothiazin-10-yl)-N-ethyl]-methylamin, [(3,4-Dimethoxyphenyl)-acetyl]-[3 (2-chlorphenothiazin-10-yl)-N-propyl]-methylamin, n (1-naphthyl)-N′[2 (3,4-dimethoxyphenyl)-ethyl]-ethyl diamine, n (1-naphthyl)-N[2 (4-methoxyphenyl)-ethyl]-ethyl diamine, n [2 (3,4-Dimethoxyphenyl)-ethyl]-n [1-naphthylmethyl]amine, n [2 (4-Methoxyphenyl)-ethyl]-n [1-naphthylmethyl]amine, [3 (10.11-Dihydro dibenzo[b,f]azepin-5-yl)-N-propyl]-[(4-methoxyphenyl)-acetyl]-methylamin, [2 (10,11-Dihydro-dibenzo[b, f]azepin-5-yl)-N-ethyl]-[2 (3,4-dimethoxyphenyl)-ethyl]methylamin, [2 (10,11-Dihydro-dibenzo[b,f]azepin-5-yl)-N-ethyl]-[2 (4-methoxyphenyl)-ethyl]-methylamin, [2 (10,11-Dihydro-dibenzo[b,f]azepin-5-yl)-N-ethyl]-[(4-methoxyphenyl)-acety-1]-methylamin, n [2 (Carbazol-9-yl)-N-ethyl]-N′[2 (4-methoxyphenyl)-ethyl]piperazin, 1 [2 (Carbazol-9-yl)-N-ethyl]-4[2 (4-methoxyphenyl)-ethyl]-3,5-dimethylpiperazin, [2 (4-Methoxyphenyl)-ethyl]-[3 (phenoxazin-10-yl)-N-propyl]-methylamin, [3 (5,6,11,12-Tetrahydrodibenzo[b,f]azocin)-N-propyl]-[3 (4-methoxyphenyl)-propyl]methylamin, n (5H-Dibenzo [A, D]cycloheptan-5-yl)-N′[2 (4-methoxyphenyl)-ethyl]-propylene diamine and [2 (Carbazol-9-yl)-N-ethyl]-[2(4-methoyphenyl)-methoxyphenyl)-ethyl]methylamine, as described in WO2000 EP04738 20000524 herein incorporated by reference. L-carnitine id also contemplated for use according to the invention (see, U.S. Pat. No. 6,114,385, herein incorporated by reference). Other suitable compounds include silymarin, 1-phenyl-2-decanoylaminon-3-morpholino-1-propanol, 1-phenyl-2-hexadecanoylaminon-3-pyrrolidino-1-propanol, Scyphostatin, L-carnitine, glutathione, and human milk bile salt-stimulated lipase (see, U.S. Pat. No. 6,663,850 herein incorporated by reference).

In some preferred embodiments, ceramide levels may be reduced by myriocin, cycloserine, Fumonisin B tyclodecan-9-xanthogenate (D609), PPMP, methylthiodihydroceramide, propanolol, resveratrol and other agents as described in U.S. Patent Application Publication No. 20050182020 or U.S. Patent Application Publication No. 20100086543, published on Apr. 8, 2010, or U.S. Patent Application Publication No. 20100204162, published on Aug. 12, 2010, all of which are herein incorporated by reference particularly with respect to the ceramide modulators of various kinds (including nucleic acids, anti-sense nucleic acids, ribozymes, anti-sense RNAs, and siRNAs, antibodies which target enzymes involved in ceramide biosynthesis) disclosed therein. Agents comprised of polypeptides sequences have also been shown to reduce ceramide levels as described in U.S. Pat. No. 7,037,700 and herein incorporated by reference.

Other ceramide modulators for use according to the invention can also include the following SPT inhibitors:

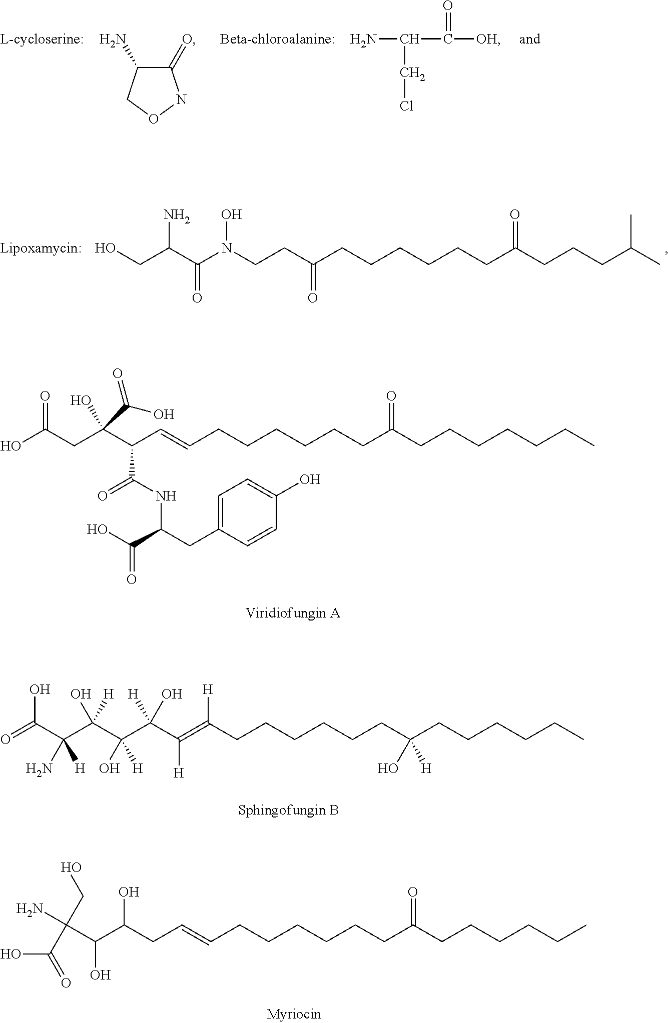

As well as the following compounds:

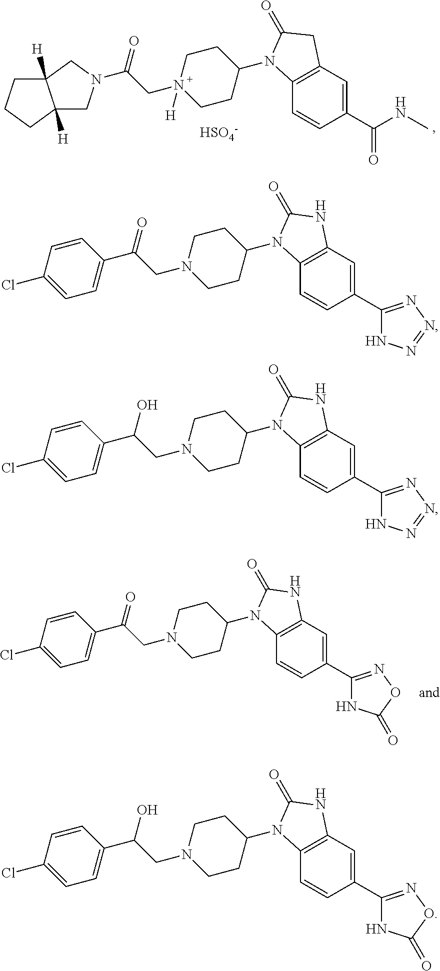

Also contemplated for use according to the present invention are deoxy-sphingolipid blockers. These compounds are compounds or substances capable of inhibiting SPT or capable of competing with the natural reactants leading to deoxy-sphingolipids in the SPT pathway (e.g., L-alanine and glycine). (See, PCT Patent Application Publication No. WO2011/104298 which is incorporated herein by reference in its entirety and particularly with respect to such agents). Blockers for use according to the invention, for instance, are L- and D-serine, D-alanine and analogues thereof (see, Kayoko Kanda et al. (Journal of General Microbiology (1988), 134, 2747-2755), Woese Cr. et al (J Bacteriol. 1958 December 76(6): 578-88) and Yasuda Y. et al (Microbiol. Immunol. 1985; 29(3): 229-41). Accordingly, blockers for use according to the invention include L-serine, D-serine, D-alanine, D-threonine, O-methyl-DL-sehne, sphingofungin B, cycloserine, myriocin, β-chloroalanine, lipoxamycin and viridofungin A, and combinations thereof. Additionally, nucleic acids, anti-sense nucleic acids, ribozymes, anti-sense RNAs, and siRNAs, antibodies which target enzymes involved in ceramide biosynthesis are also contemplated as modulators. 3. The composition of claim 1 comprising at least one first substance capable of competing with L-alanine and glycine in the reaction catalysed by SPT and at least one second substance capable of inhibiting serine-palmitoyltransferase (SPT).

This list is non-exhaustive. One of ordinary skill in the art would appreciate that analogs or fragments of the inhibitors included herein would similarly be inhibitory. In addition to the agents described herein are agents that decrease ceramide pathway metabolic enzymes, or increase ceramide catabolic enzymes, including but not limited to agents, which modify, or regulate transcriptional or translational activity or which otherwise degrade, inactivate, or protect theses enzymes.

The “subject” to be treated includes any animal, including, but not limited to, mammals (e.g., rat, mouse, cat, dog) including humans to which a treatment is to be given. “Mammal” includes humans and non-human mammals (e.g., dogs, cats, rabbits, cattle, horses, sheep, goats, swine, rats, mice, and primates).

The term “effective amount” means a dosage sufficient to produce a given result with respect to the indicated disorder or condition. In the case of therapeutic methods, the result may comprise a subjective or objective improvement in the recipient of the dosage.

The terms “treatment”, “therapy” and the like include, but are not limited to, methods and manipulations to produce beneficial changes in a recipient's health status or reduce or prevent a pathology induced by an amphetamine drug such as methamphetamine and mediated by increased ceramide levels and/or apoptosis. Preventing or reducing the deterioration of a recipient's status is also included by the term. Therapeutic benefit includes any of a number of subjective or objective factors indicating a beneficial response or improvement of the toxicity being treated as discussed herein.

Pharmaceutical compositions are also provided by the invention. A pharmaceutical composition comprising a therapeutically effective amount of an amphetamine drug; and one or more of a therapeutically effective amounts of an agent for use according to the invention. In some embodiments, the agent is an inhibitor of ceramide biosynthesis or apoptosis; and a pharmaceutically acceptable carrier. For example, the agent can be a compound (nucleic acid, antibody, small or large molecule) that inhibits ceramide biosynthesis by targeting at least one ceramide-biosynthetic enzyme selected from the group consisting of a sphingomyelinase, serine palmitoyltransferase, 3-ketosphinganine reductase, ceramide synthase, dihydroceramide desaturase, and combinations thereof. In other embodiments, the agent that inhibits ceramide biosynthesis comprises a compound selected from the group consisting of Fumonisin B1 (FB1), tyclodecan-9-xanthogenate (D609), myriocin and combinations thereof.

Pharmaceutically acceptable carriers to be used in formulating a compound for use according to the invention are determined in part by the particular compound being administered, as well as by the particular method used to administer the composition. Accordingly, there are a wide variety of suitable formulations of pharmaceutical compositions of the present invention (see, e.g., Remington's Pharmaceutical Sciences, 20^{th} ed., 2003, supra).

Formulations suitable for oral administration can consist of (a) liquid solutions, such as an effective amount of the packaged nucleic acid suspended in diluents, such as water, saline or PEG 400; (b) capsules, sachets or tablets, each containing a predetermined amount of the active ingredient, as liquids, solids, granules or gelatin; (c) suspensions in an appropriate liquid; and (d) suitable emulsions. Tablet forms can include one or more of lactose, sucrose, mannitol, sorbitol, calcium phosphates, corn starch, potato starch, microcrystalline cellulose, gelatin, colloidal silicon dioxide, talc, magnesium stearate, stearic acid, and other excipients, colorants, fillers, binders, diluents, buffering agents, moistening agents, preservatives, flavoring agents, dyes, disintegrating agents, and pharmaceutically compatible carriers. Lozenge forms can comprise the active ingredient in a flavor, e.g., sucrose, as well as pastilles comprising the active ingredient in an inert base, such as gelatin and glycerin or sucrose and acacia emulsions, gels, and the like containing, in addition to the active ingredient, carriers known in the art.

The compound, alone or in combination with other suitable components, can be made into aerosol formulations (i.e., they can be “nebulized”) to be administered via inhalation. Aerosol formulations can be placed into pressurized acceptable propellants, such as dichlorodifluoromethane, propane, nitrogen, and the like.

Suitable formulations for rectal administration include, for example, suppositories, which consist of the packaged nucleic acid with a suppository base. Suitable suppository bases include natural or synthetic triglycerides or paraffin hydrocarbons. In addition, it is also possible to use gelatin rectal capsules which consist of a combination of the compound of choice with a base, including, for example, liquid triglycerides, polyethylene glycols, and paraffin hydrocarbons.

Formulations suitable for parenteral administration, such as, for example, by intraarticular (in the joints), intravenous, intramuscular, intratumoral, intradermal, intraperitoneal, and subcutaneous routes, include aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. In the practice of this invention, compositions can be administered, for example, by intravenous infusion, orally, topically, intraperitoneally, intravesically or intrathecally. Parenteral administration, oral administration, and intravenous administration are the preferred methods of administration. The formulations of compounds can be presented in unit-dose or multi-dose sealed containers, such as ampules and vials.

Injection solutions and suspensions can be prepared from sterile powders, granules, and tablets of the kind previously described. Cells transduced by nucleic acids for ex vive therapy can also be administered intravenously or parenterally as described above.

The pharmaceutical preparation is preferably in unit dosage form. In such form the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form. The composition can, if desired, also contain other compatible therapeutic agents.

Preferred pharmaceutical preparations deliver one or more compounds for use according to the invention, optionally in combination with one or more other agents (e.g., an amphetamine drug). Sustained release formulations of compounds for use according to the invention are also contemplated.

In therapeutic use for the treatment of amphetamine drug induced toxicity, the compounds utilized in the pharmaceutical method of the invention are administered at the initial dosage of about 0.001 mg/kg to about 1000 mg/kg daily. A daily dose range of about 0.01 mg/kg to about 500 mg/kg, or about 0.1 mg/kg to about 200 mg/kg, or about 1 mg/kg to about 100 mg/kg, or about 10 mg/kg to about 50 mg/kg, can be used. The dosages, however, may be varied depending upon the requirements of the patient, the severity of the condition being treated, and the compound being employed. For example, dosages can be empirically determined considering the particular patient. The dose administered to a patient, in the context of the present invention should be sufficient to effect a beneficial therapeutic response in the patient over time. Determination of the proper dosage for a particular situation is within the skill of the practitioner. Generally, treatment can be initiated with smaller dosages which are less than the optimum dose of the compound. Thereafter, the dosage is increased by small increments until the optimum effect under circumstances is reached. For convenience, the total daily dosage may be divided and administered in portions during the day, if desired.

The pharmaceutical preparations are typically delivered to a mammal, including humans and non-human mammals.

### Methods for Testing Compounds for Use According to the Invention

The invention also provides methods of testing a compound for use according to the invention comprising the steps of: (a) contacting in vivo or in vitro a mammalian cell(s) with an amphetamine drug and the compound to be tested and determining whether the contacting reduces the formation of a deoxy-sphingolipid, a ceramide, or reduces apoptosis in the cell(s) as compared to a control mammalian cell(s) contacted with the amphetamine drug and not the test compound. Ceramide species to monitor include, but are not limited to, Cer(16:0), Cer(16:1), Cer(18:0), Cer(20:0), Cer(20:1), Cer(24:1); and dihydroceramide species including, but not limited to, DHCer(16:0), DHCer(16:1), DHCer(18:0), DHCer(20:0), DHCer(20:1), DH Cer(24:1). The invention also provides methods of testing a compound for use according to the invention comprising the steps of: (a) administering the test compound to a mammal also treated or to be treated with an amphetamine drug dose which increases ceramide levels (e.g., a toxic dose of the amphetamine drug) in the mammal and determining whether the administered test compound reduces the formation of a deoxy-sphingolipid, a ceramide, or reduces apoptosis, and/or reduces an adverse health effect in the mammal as compared to a control mammal treated with the amphetamine drug and not the test compound. Ceramide species to monitor include, but are not limited to, Cer(16:0), Cer(16:1), Cer(18:0), Cer(20:0), Cer(20:1), Cer(24:1); and dihydroceramide species including, but not limited to, DHCer(16:0), DHCer(16:1), DHCer(18:0), DHCer(20:0), DHCer(20:1), DH Cer(24:1). In some in such methods, the adverse effect is on an affected organ as described above for amphetamine drug-induced toxicities. In some embodiments, the subject is as described above (e.g., a human, a primate, or a rodent (rat, mouse). In some embodiments, samples from the test and control subject are taken and analyzed for levels of a lipid of the ceramide pathway whose levels are affected by the methamphetamine treatment. The sample can be a tissue sample taken from one or more of an affected organ, blood, or urine.

### Monitoring for Amphetamine Drug Toxicity and Amphetamine Drug Administration.

In a further aspect, a new set of lipid biomarkers (e.g., ceramides, Cer(16:0), Cer(16:1), Cer(18:0), Cer(20:0), Cer(20:1), Cer(24:1); and dihydroceramide species including, but not limited to, DHCer(16:0), DHCer(16:1), DHCer(18:0), DHCer(20:0), DHCer(20:1), DH Cer(24:1)) for use in assessing, diagnosing and monitoring for any toxicity induced by amphetamine drug is provided. Accordingly, the invention provides a method for monitoring with ease, low-invasiveness and low-cost peripheral biomarkers of amphetamine toxicity (i.e., ceramides), which find applications in the following areas: (1) prophylactic and diagnostic screening in a large population of subjects; (2) leading to a more accurate diagnostic tool, especially if used in combination with other clinical parameters; (3) assessing drug response in asymptomatic patients; (4) serving as a secondary outcome measure in clinical trials of symptomatic patients, and (5) deciding if further development of a treatment should be stopped if not likely to be effective. In this aspect, samples of tissue are taken from a subject having been administered an amphetamine drug or suspected of having been administered an amphetamine drug and the samples are then analyzed for the amount of one or more lipids of the ceramide pathway. The amount of the analyzed lipid is then compared to that for a control or reference population not having been exposed to the amphetamine drug. Alternatively, or in addition, the levels of the analyzed lipid can be tracked over time by repeated sampling of the individual subject, and the trend of the lipid levels over time compared. Elevated levels of the analyzed lipid compared to controls levels being indicative of an amphetamine toxicity and/or likely use or continued use of amphetamine by the subject. The sample can be a tissue sample taken from one or more of an affected organ, blood, saliva, or urine. In some embodiments, the monitoring is repeated over time to track the health status or continued use of an amphetamine drug by the subject. In some further embodiments, the subject (e.g., an amphetamine-type drug user, a person suspected of same) identified to have an elevated ceramide lipid levels as shown by the above assessing, diagnosing or monitoring is further treated with a modulator of ceramide metabolism or apoptosis according to the methods of the invention. In still some further embodiments of same the modulator dosing is adjusted or ended according to the results of monitoring the ceramide lipid levels over time. In some additional embodiments of any of the above, the same or additional samples from the subject are also tested for the presence of an amphetamine-type drug or its metabolites.

The following examples are provided for illustrative purposes, and are not intended to limit the scope of the invention as claimed herein. Any variations in the exemplified articles and/or methods which occur to the skilled artisan are intended to fall within the scope of the present invention.

### EXAMPLE

### Targeting Ceramide in Amphetamine Toxicity

### Elevated Ceramide Levels in Rats Self-Administering Methamphetamine

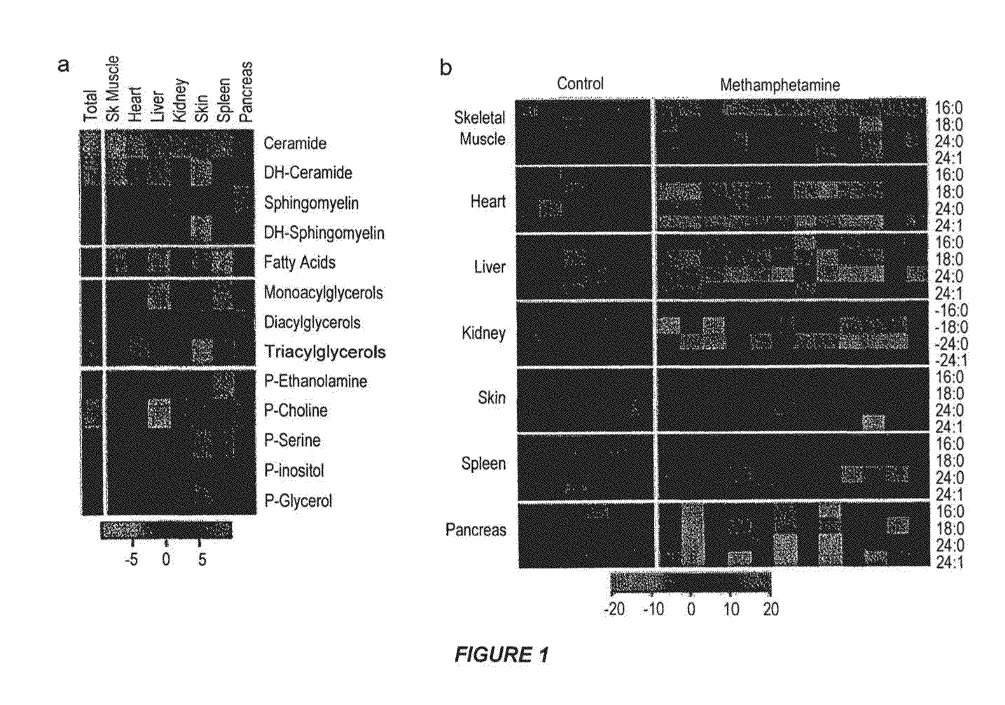

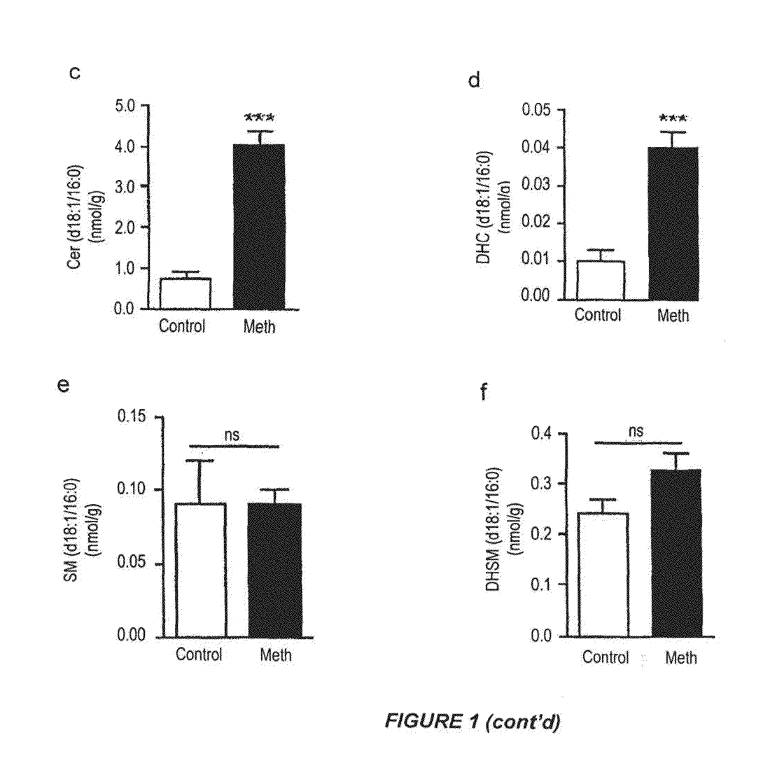

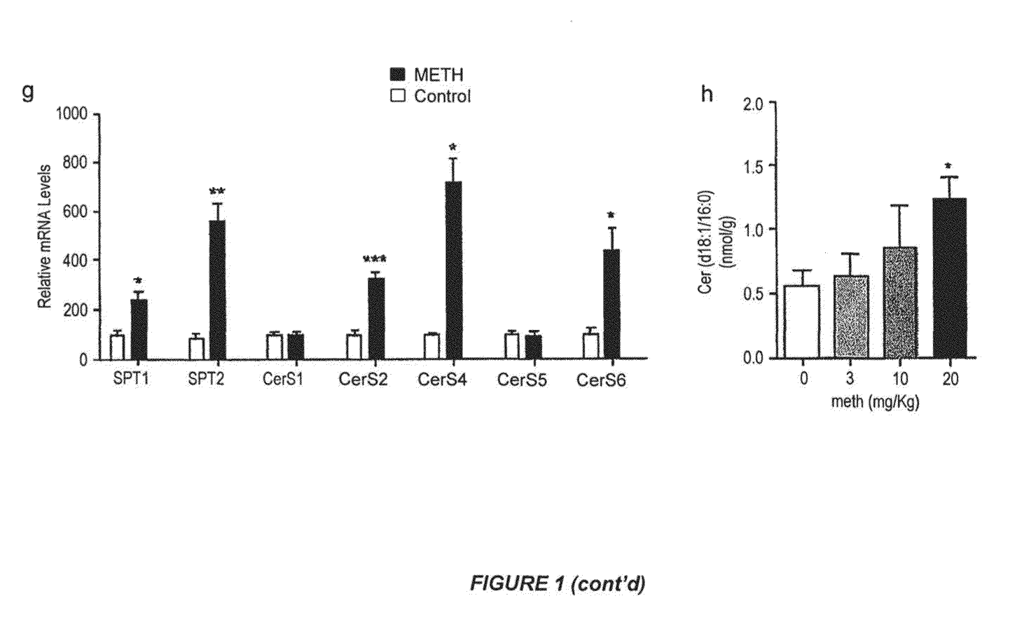

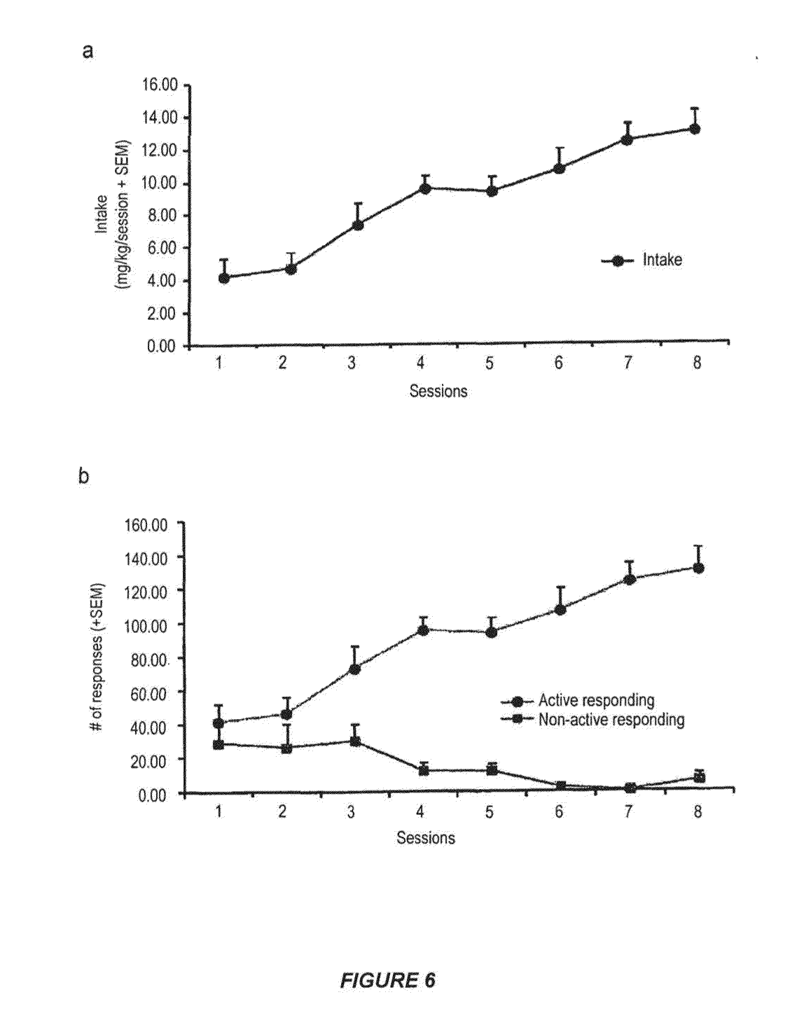

Methamphetamine self-administration in rats closely mimics the voluntary component of human drug exposure, and is characterized by high rates of drug intake (FIG. 6) [11]. To examine whether high methamphetamine intake is accompanied by abnormality in lipid profile, we conducted an unbiased lipidomic analysis of various tissues obtained from rats that self-administered methamphetamine and compared these to controls. Lipids were extracted and analyzed by LC/MSⁿ for the major lipid classes. To facilitate visual inspection of broad regions of interest in the lipidome, LC/MSⁿ data were processed statistically using heat-maps. This survey revealed that multiple ceramide species were substantially increased in methamphetamine-exposed rats, compared to controls (FIG. 1a). The largest increase in ceramide levels were seen in the skeletal muscle, heart and liver (FIG. 1 b). To investigate whether the elevation in ceramide levels in rats self-administering methamphetamine resulted from a stimulation of ceramide biosynthesis we conducted a focused lipidomic analysis on skeletal muscle. Methamphetamine self-administration was associated with a 4-fold increase in ceramide (d18:1/16:0) (FIG. 1c) and dihydroceramide (d18:1/16:0) (FIG. 1d). By contrast the levels of sphingomyelin (d18:1/16:0) (FIG. 1e) and dihydrosphingomyelin (d18:1/16:0) (FIG. 1f), which generate ceramide through the degradative pathway, were unchanged. This increase in ceramide was accompanied by increased serine palmitoyltransferase (SPT) and ceramide synthase (CerS) gene expression (FIG. 1g) and increased CerS activity (data not shown). These results indicate that methamphetamine self-administration in rats increased de novo ceramide biosynthesis in skeletal muscle.

As prolonged methamphetamine self-administration exerts negative health effects we tested the acute effects of methamphetamine in rats. Intraperitoneal administration of methamphetamine (3, 10, and 20 mg/kg) caused a dose-dependent increase in d18:1/16:0 ceramide levels in rat skeletal muscle (FIG. 1h), and other peripheral tissues (data not shown). As seen with methamphetamine self-administration, non-contingent methamphetamine administration increased CerS6 gene expression, one of the main enzymes responsible for generating d18:1/16:0 ceramide [12], (data not shown) and increased CerS activity (data not shown).

### D-Methamphetamine Enhances De Novo Ceramide Biosynthesis in Primary Mouse Embryonic Fibroblasts

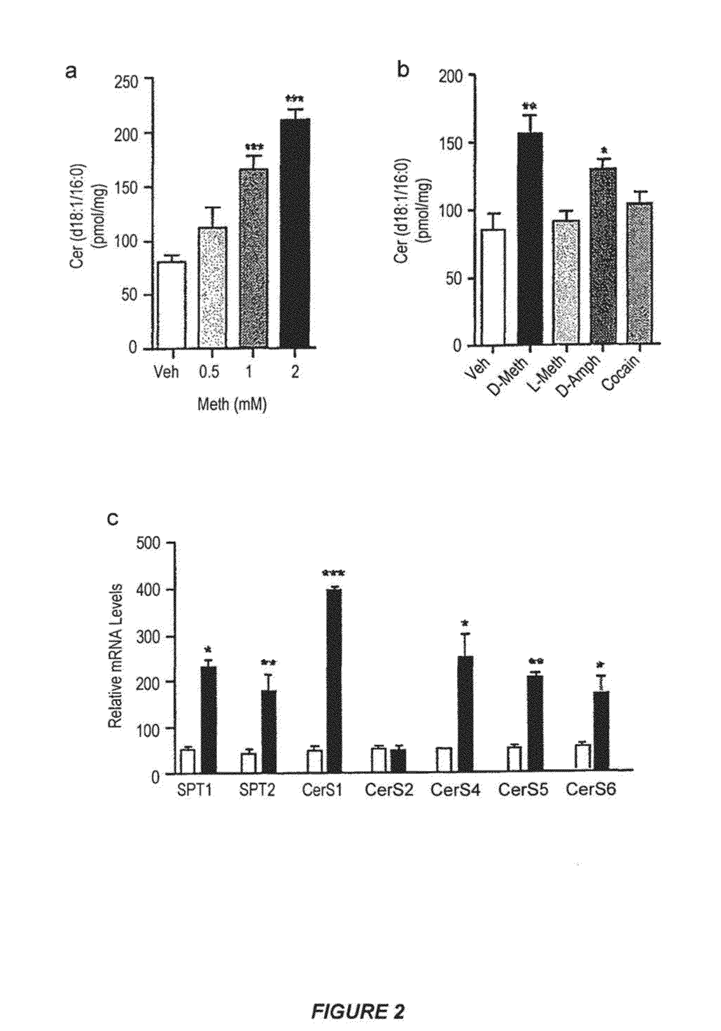

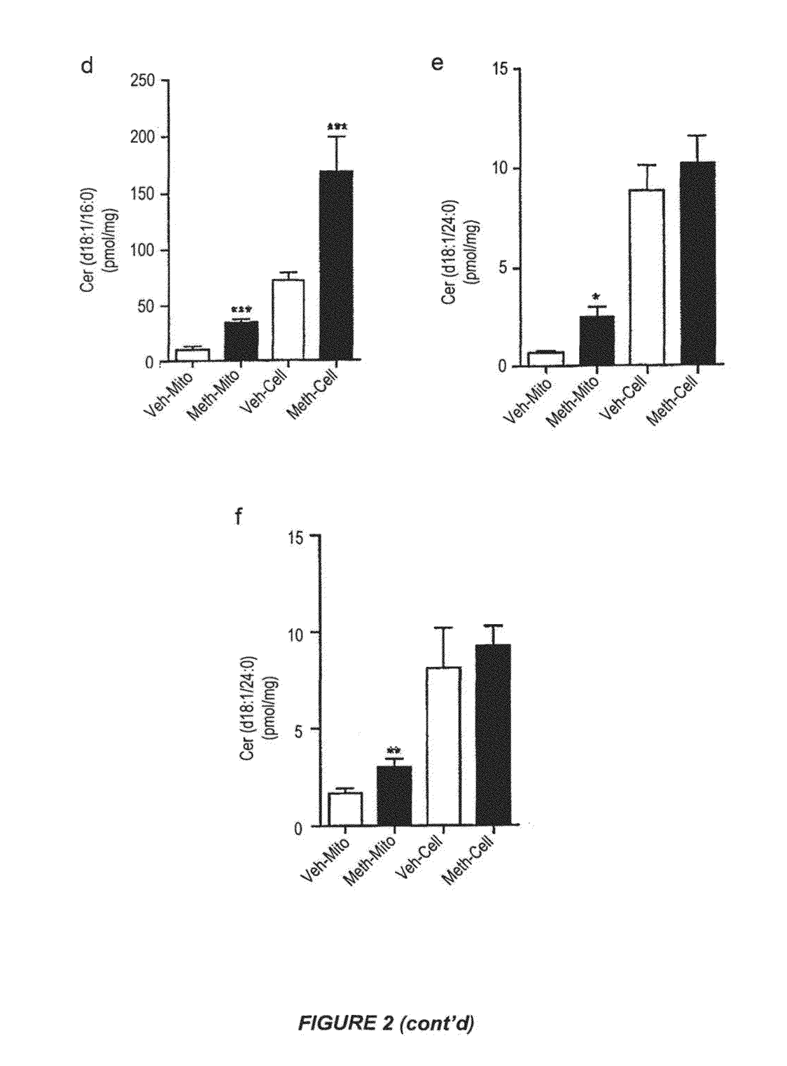

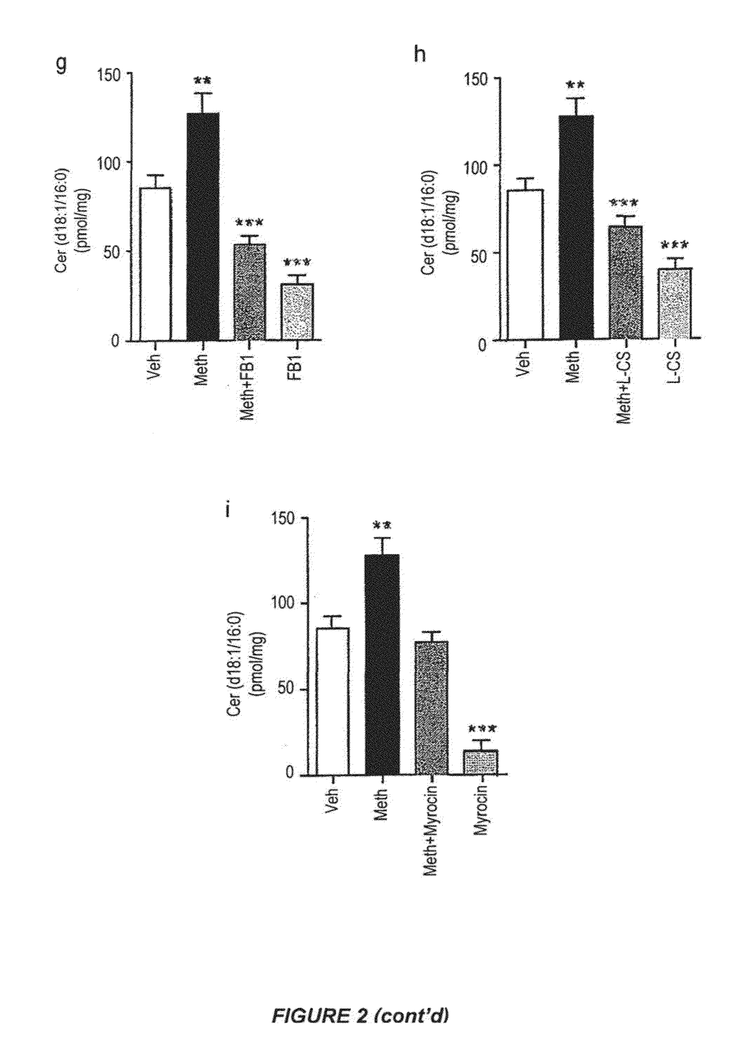

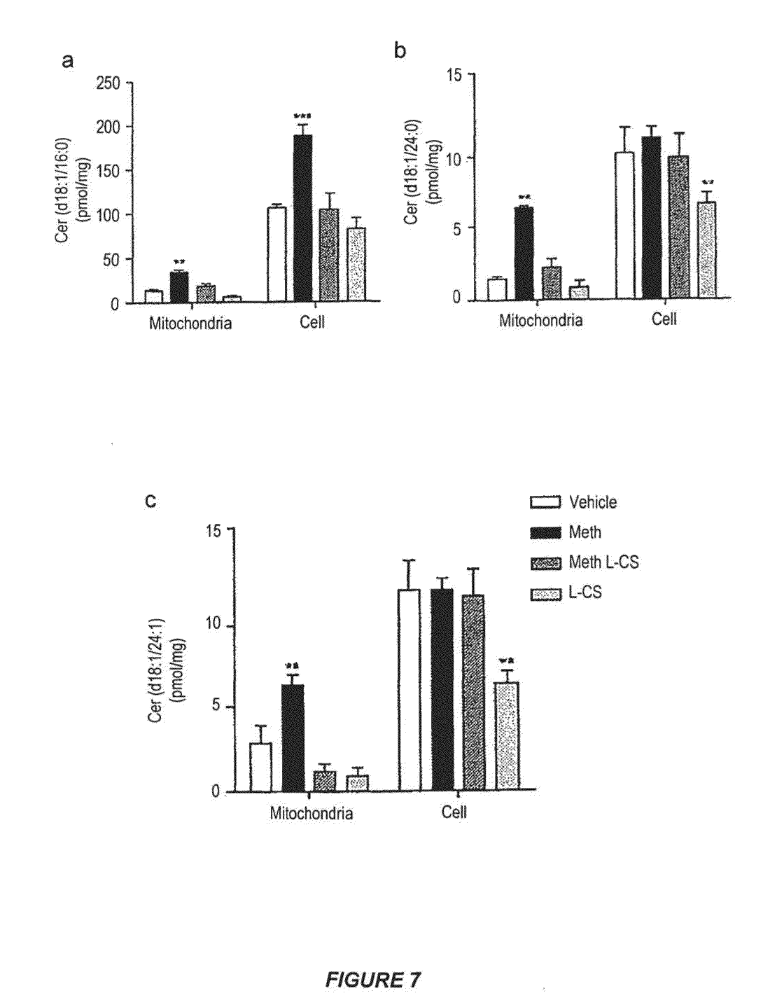

As a further test for the ability of methamphetamine to alter ceramide production we used primary mouse embryonic fibroblasts (MEF). Incubation of MEF with methamphetamine increased ceramide (d18:1/16:0) in a dose-dependent manner (FIG. 2a). The effect of methamphetamine was stereospecific; the non-toxic enantiomer 1-methamphetamine has no such effect (FIG. 2b). Interestingly, d-amphetamine also produced a modest but statistically significant (p<0.014) increase in ceramide content (FIG. 2b). The increased ceramide levels induced by methamphetamine were accompanied by increased mRNA levels of the genes involved de novo ceramide biosynthesis (FIG. 2c). The main sight of de novo ceramide biosynthesis is the endoplasmic reticulum; however recent work has identified CerS enzymes in the mitochondria, indicating that some de novo ceramide biosynthesis also occurs in the mitochondria. Lipid analysis of isolated mitochondria from MEF treated with methamphetamine (1 mM) showed a unique pattern of ceramide increase in which in addition to increases in short chain ceramide (d18:1/16:0) (FIG. 2d) we also found a mitochondrial specific increase in long chain ceramides (d18:1/24:0 and d18:1/24:1) (FIG. 2e, f). The increase in long chain ceramides specifically in the mitochondria can result in increased mitochondrial dysfunction and the production of excess reactive oxygen species. Further, 30 μM L-cycloserine (L-CS), an inhibitor of SPT [13], was able to block methamphetamine induced increases in ceramide in primary MEF (FIG. 2h, i) and in isolated mitochondria from primary MEF (FIG. 7). Similar results were obtained using 50 μM Fumonisin B1 (FB1), a potent inhibitor of CerS and 10 μM myrocin, a structurally distinct SPT inhibitor [14](FIG. 2g). Overall, these findings indicate that methamphetamine treatment induced a stereospecific activation of the de novo biosynthetic pathway in MEF cells.

### Methamphetamine Accelerates Senescence in Primary Mouse Embryonic Fibroblasts

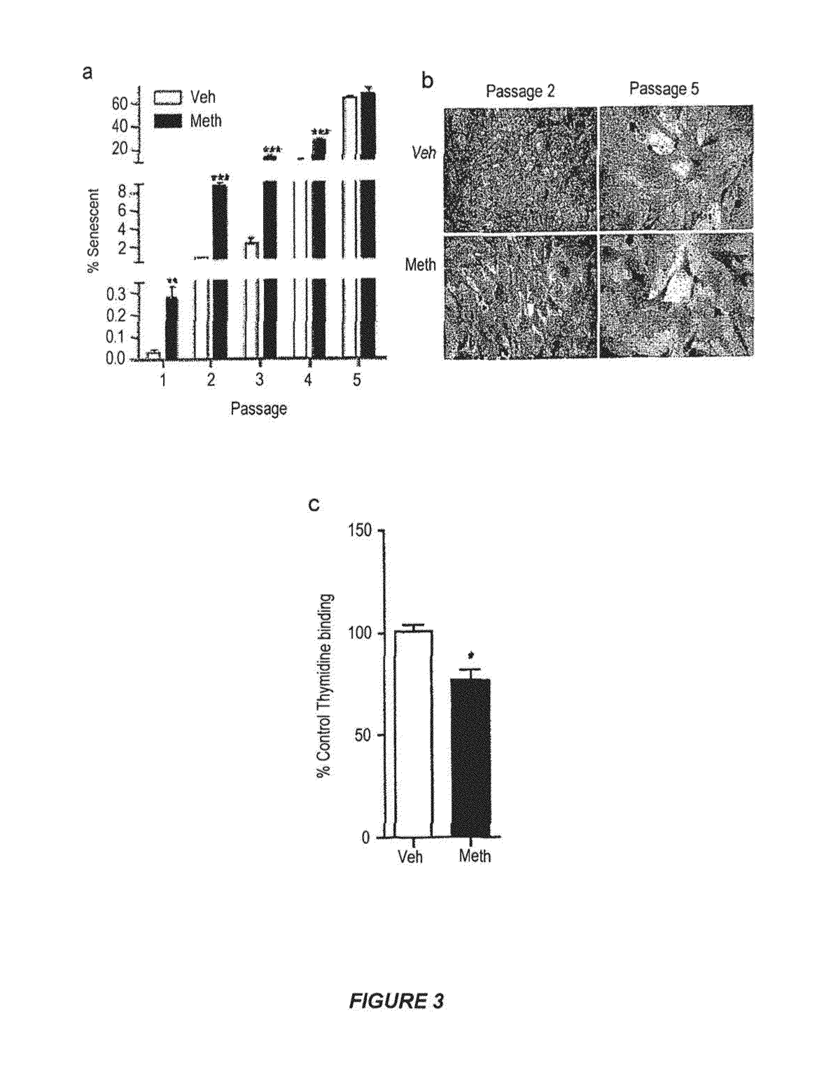

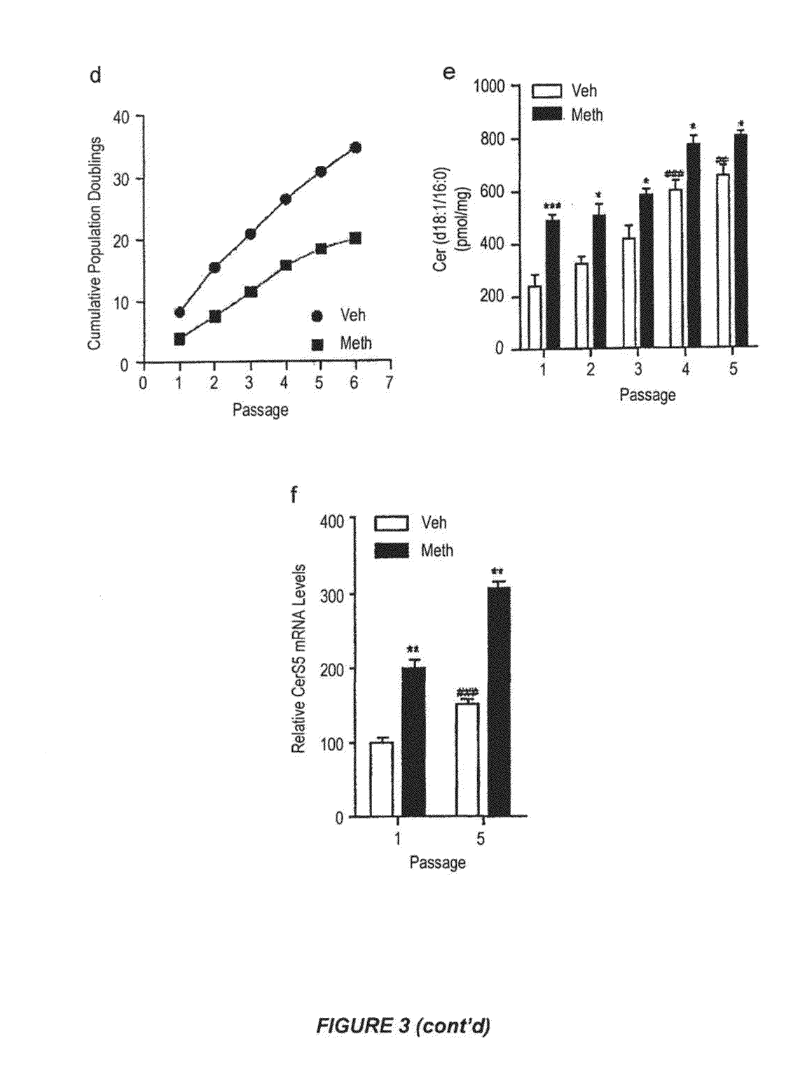

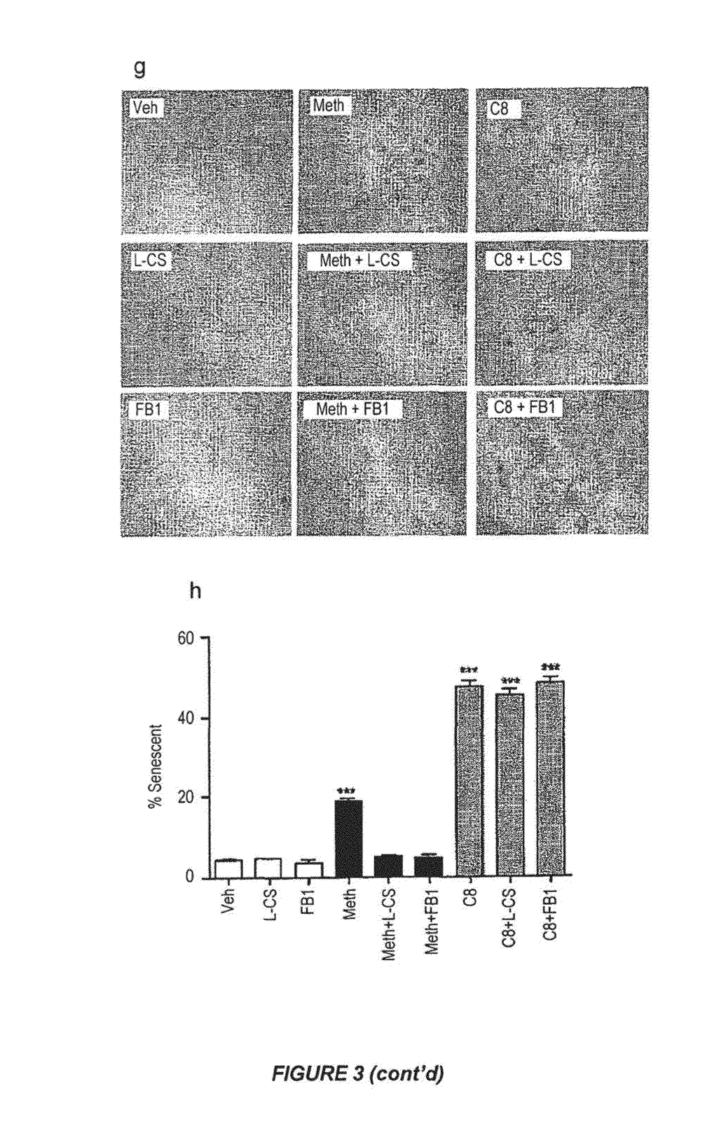

Primary cells in culture exhibit a finite proliferative lifespan with a limited capacity to undergo population doubling before they stop dividing [13, 14]. Limitations in proliferative capacity correlate with the age of the organism and the life expectancy of the species from which the cells are obtained from; such that the older the age or the shorter the lifespan, the lesser the ability of the cells to undergo population doubling [15]. Cells which can no longer replicate are said to be senescent, a state where normal somatic cells lose their replicative capacity resulting in an irreversible growth arrest. We treated MEFs with methamphetamine (1 mM) for 48 h and measured expression of the well-established histochemical marker of senescence, senescence associated β-galactosidase (SA-β-gal). Methamphetamine exposure increased the number of SA-β-gal positive cells from passage 1 to 4 compared to vehicle-treated cells (FIG. 3a). At passage 5, when MEF cells begin to reach the end of their replicative capacity, methamphetamine did not further increase the number of SA-β-gal positive cells (FIG. 3a). The largest methamphetamine induced elevation in the number of SA-β-gal positive cells was seen at passage 3, with a greater than 4-fold increase versus non-treated cells. Senescent cells also display an enlarged and flattened morphology as compared to actively replicating cells. Following a 48 h methamphetamine treatment (I mM) at passage 2 MEF display morphological changes typical of senescent phenotype (FIG. 3b). Consistent with the SA-β-gal data, at passage 5 both treated and untreated cells displayed a senescent morphology as the MEF naturally reached the end of their replicative lifespan. We then examined the effect of methamphetamine treatment on replicative capacity. DNA synthesis, measured using [³H]-thymidine binding, decreased following 48 h of methamphetamine treatment (1 mM) (FIG. 3c), as did the number of population doublings compared to control cells (FIG. 3d). To validate that ceramide might be mediating the induction of senescence we looked at the levels of ceramide from passage 1 to 5 and found a steady increase in ceramide levels (FIG. 3e) as well as in increase in CerS5 (FIG. 3f). Given that methamphetamine increased ceramide levels and that ceramide plays a large role in the induction of senescence, we hypothesized that the blockage of ceramide biosynthesis would ameliorate methamphetamine induced senescence. To test this hypothesis we treated MEFs with methamphetamine (1 mM) in the presence of structurally distinct inhibitors of de novo ceramide biosynthesis, L-cycloserine (L-CS) and fumonisin B1 (FB1). Treatment with L-CS (30 μM) or FB1 (50 μM) suppressed SA-β-gal expression induced by methamphetamine (FIG. 3g,h). In contrast, neither L-CS nor FB1 blocked the expression of SA-β-gal by the cell-permeable ceramide analog C8 (FIG. 3g,h). The results suggest that methamphetamine accelerates cell senescence and that blocking ceramide biosynthesis can decrease senescence induced by methamphetamine.

### Methamphetamine Activates Pro-Inflammatory and Pro-Senescent Genes Through an NF-κB Dependent Mechanism

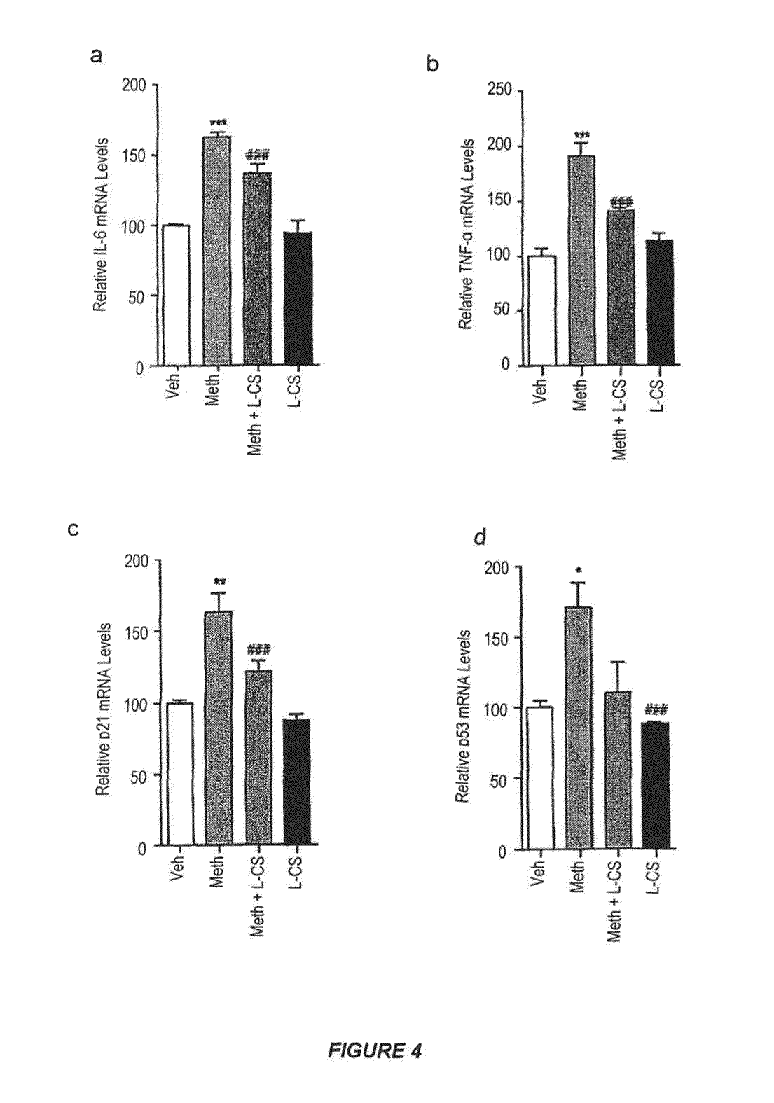

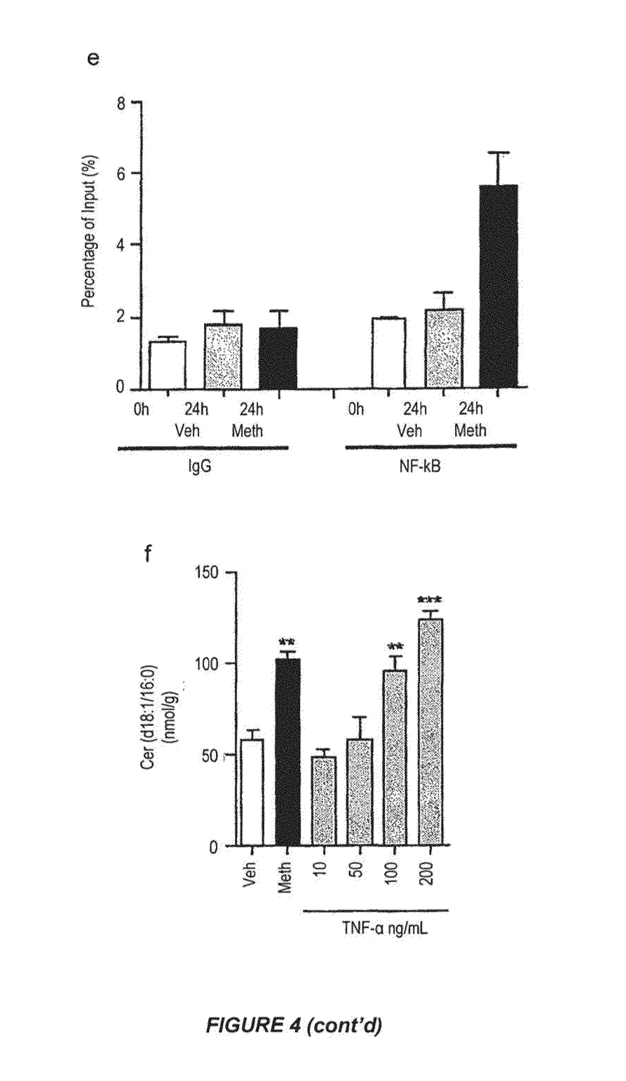

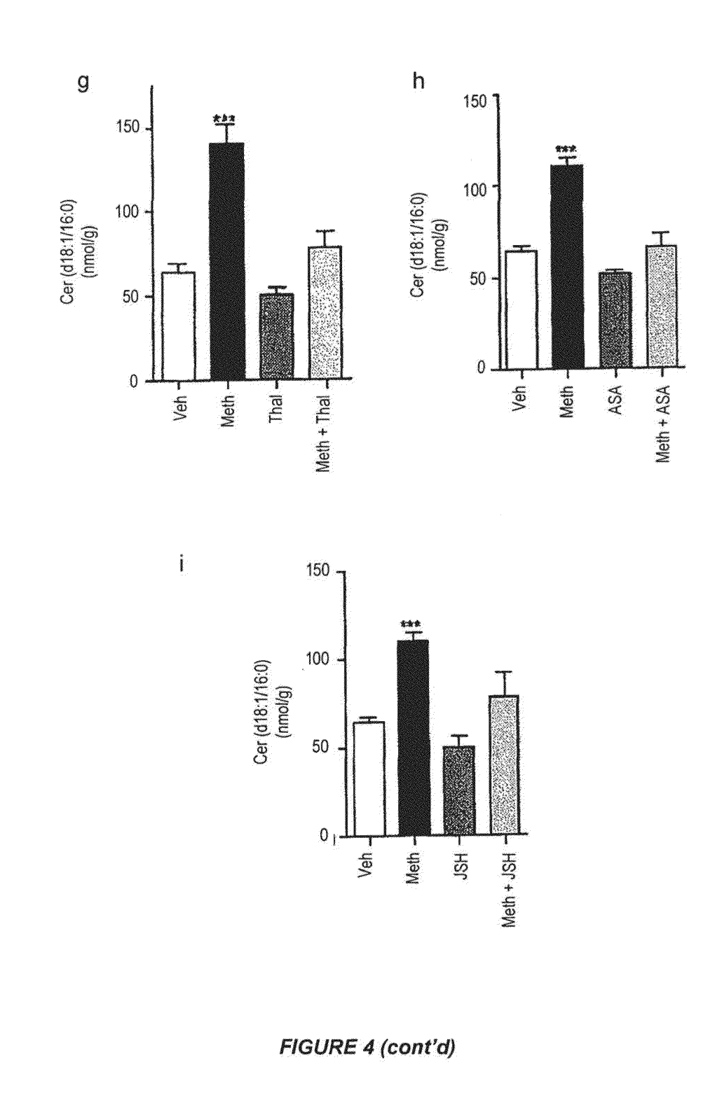

To understand how methamphetamine induces senescence in MEF we analyzed the gene expression of well-known markers of aging which contribute to inflammation and senescence. In addition to morphological and histochemical changes, senescent cells display changes in many cell cycle-regulating genes such as p15, p16, p23, and p53 [16, 17]. Additionally, studies on the biomarkers of aging have shown that IL-6, TNF-α and other inflammatory cytokines are the most reliable aging parameters [18]. We found that methamphetamine treatment resulted in the elevation of the transcription of inflammatory cytokines, IL-6 and TNF-alpha, as well as an increase in the cyclin dependent kinases p21 and p53 (FIGS. 4a-d). The increase in the transcription of these genes was dependent on increased cellular ceramide contents, as this increase was blocked with the co-administration of the SPT inhibitor L-CS. The activation of these genes following treatment pointed to a possible NF-κB dependent activation following methamphetamine treatment. MEFs treated with methamphetamine (1 mM) were harvested 24 hrs later and subjected to chromatin immunoprecipitation assays. We found recruitment of NF-kB subunit p65 to the TNF-α promoter (FIG. 4e) indicating that methamphetamine treatment activated NF-kB's transcriptional activity. Additionally, we were also able to mimic the effects of methamphetamine on cellular ceramide content by activating NF-κB with TNF-α treatment, suggesting that NF-kB activation is a necessary step in ceramide biosynthesis (FIG. 4f). To further test if NF-κB activation was essential for increased cellular ceramide content following methamphetamine treatment we treated cells with three distinctly different NF-κB inhibitors, JSH-23, 5′-aminosalicyclic acid and thalidomide in an attempt to block the effects of methamphetamine on de novo ceramide biosynthesis. We found that all three inhibitors of NF-κB were able to inhibit the effect of methamphetamine on MEF cells indicating that methamphetamine is dependent on the activation of this pathway (FIG. 4g-i). These data indicate that the succession of events which lead to the activation of de novo ceramide biosynthesis following methamphetamine treatment is dependent on the activation of NF-κB.

### Methamphetamine Increases the Expression of Inflammatory and Senescent Genes In Vivo

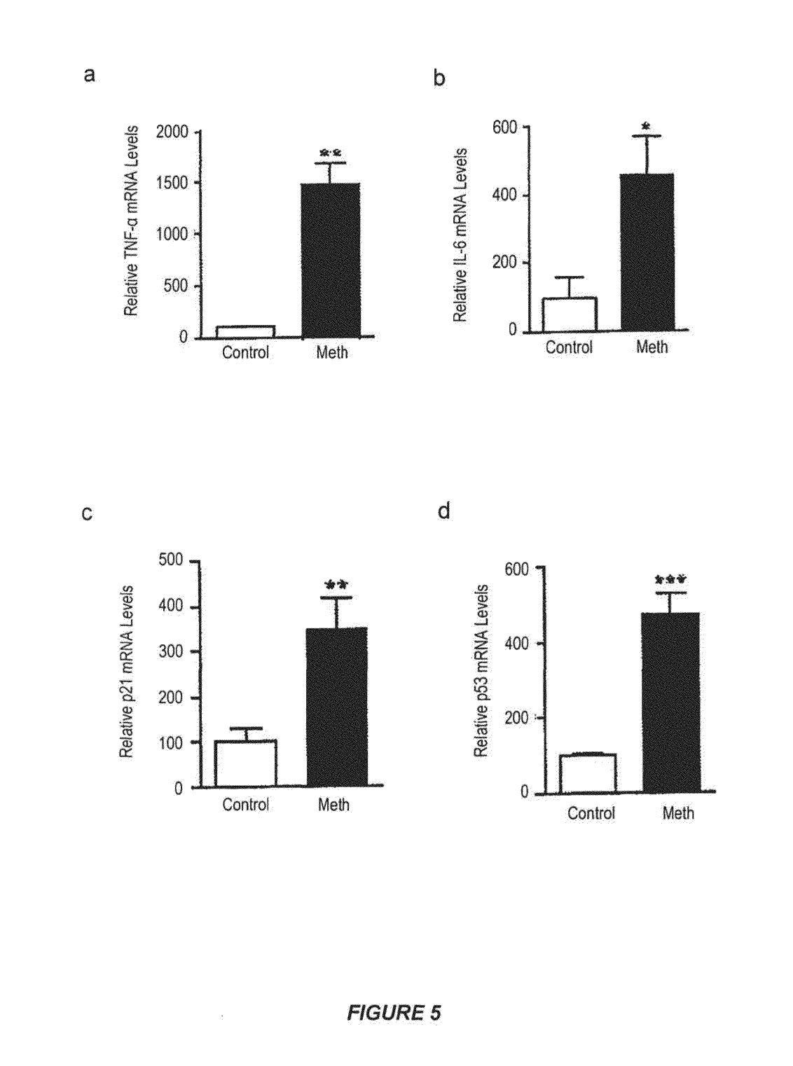

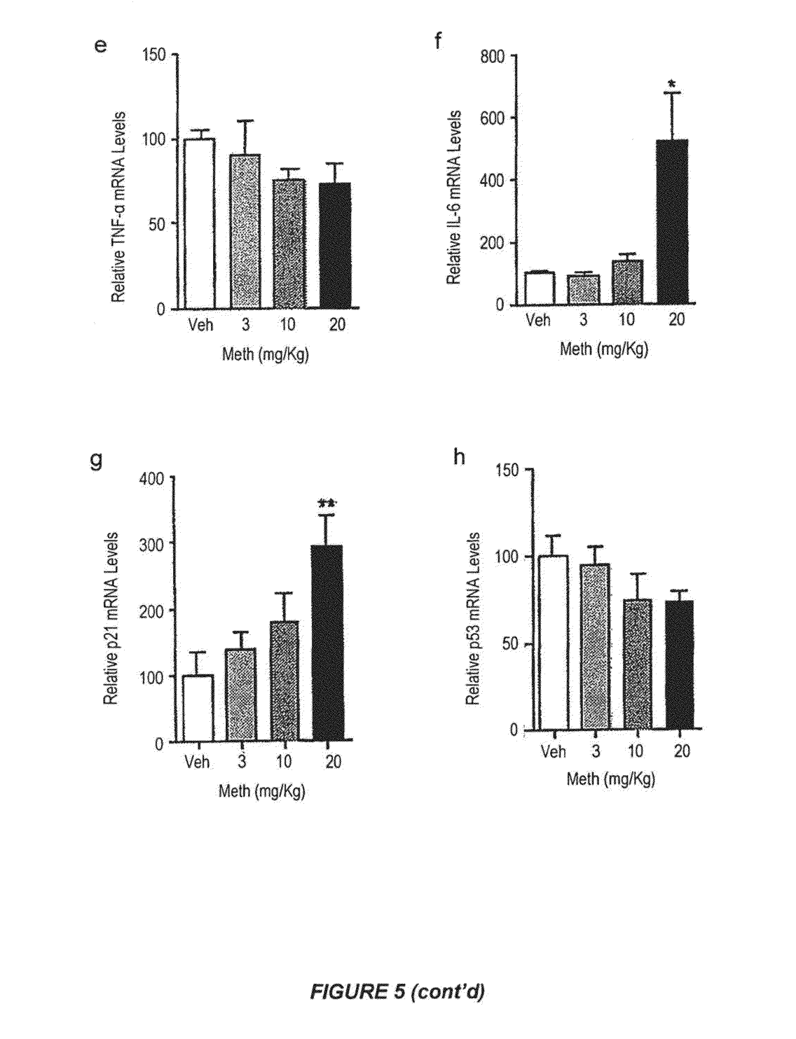

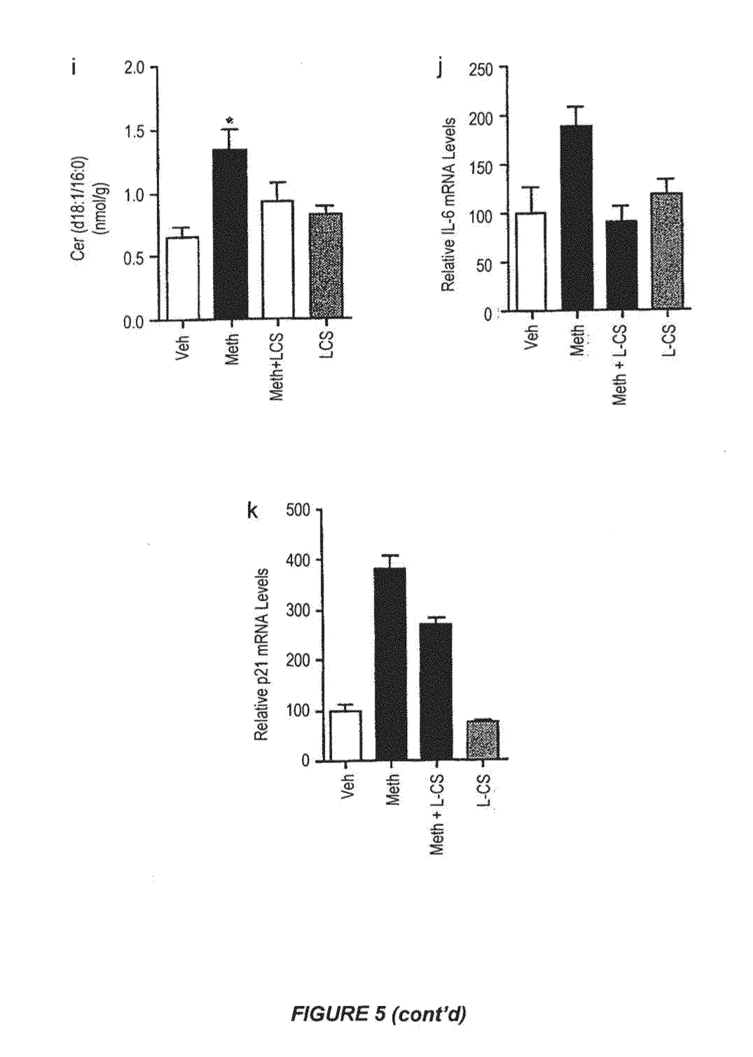

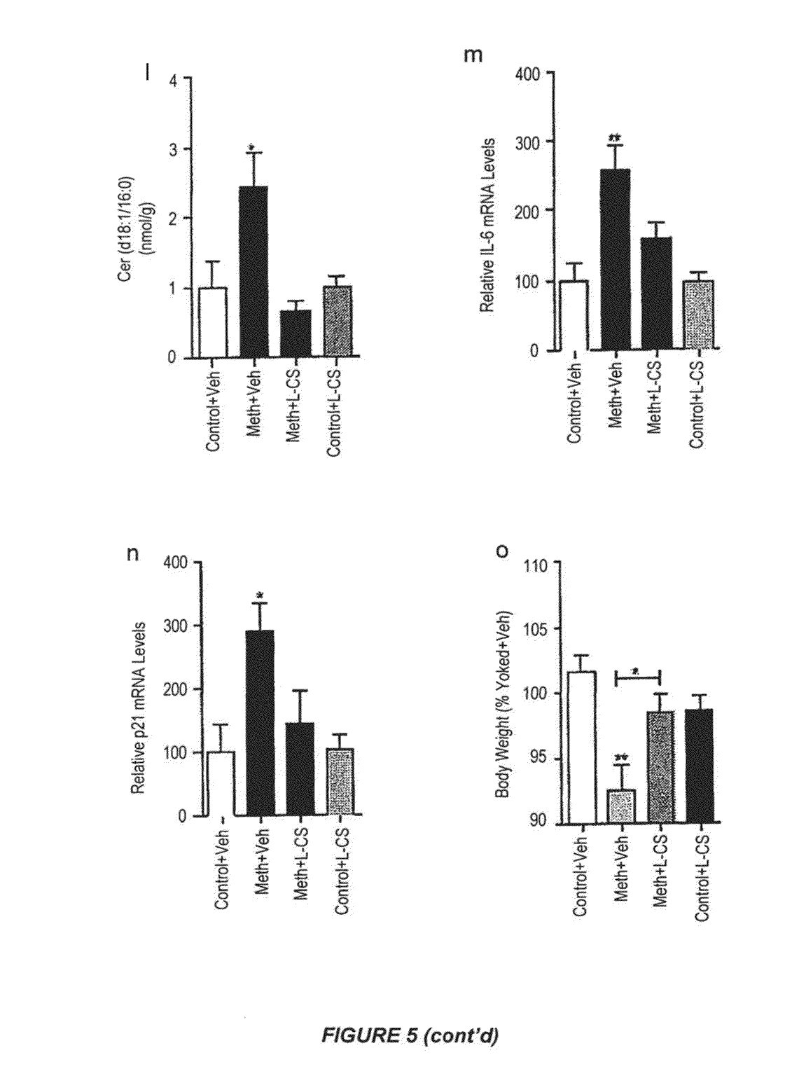

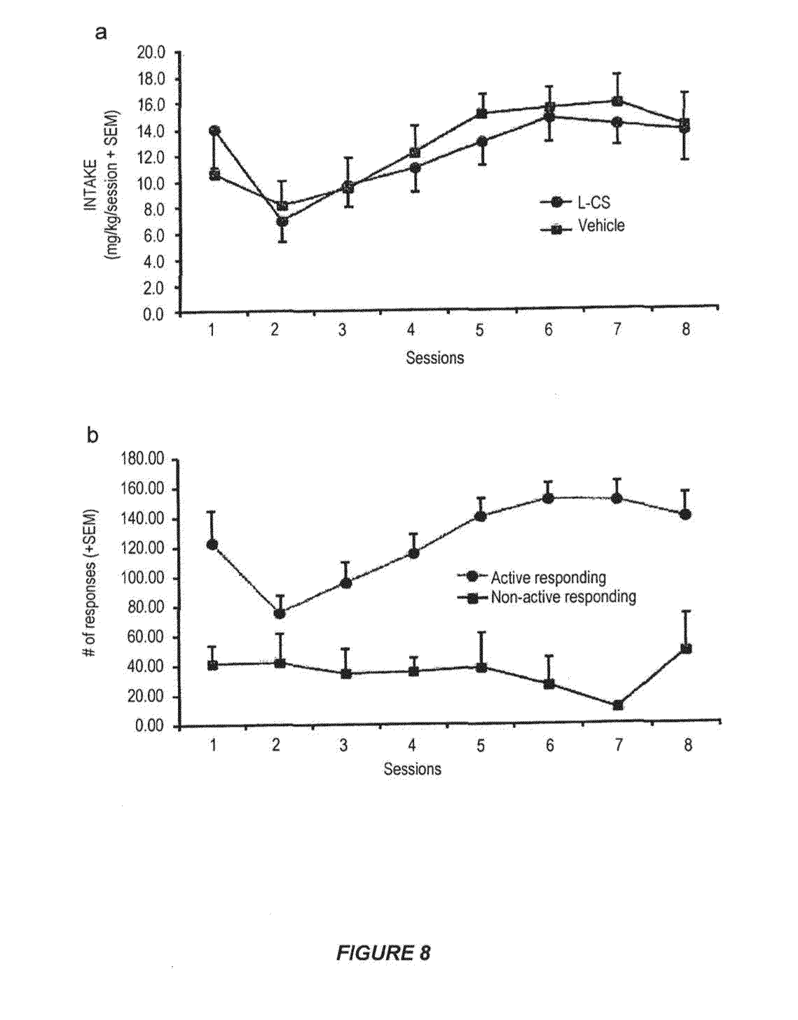

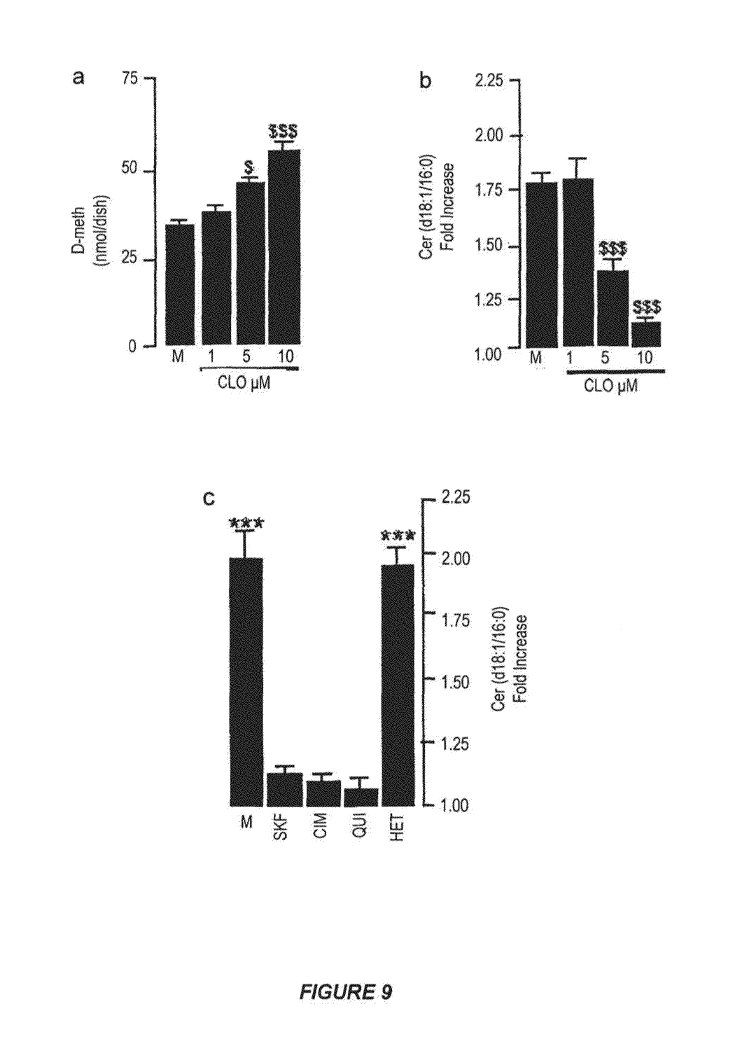

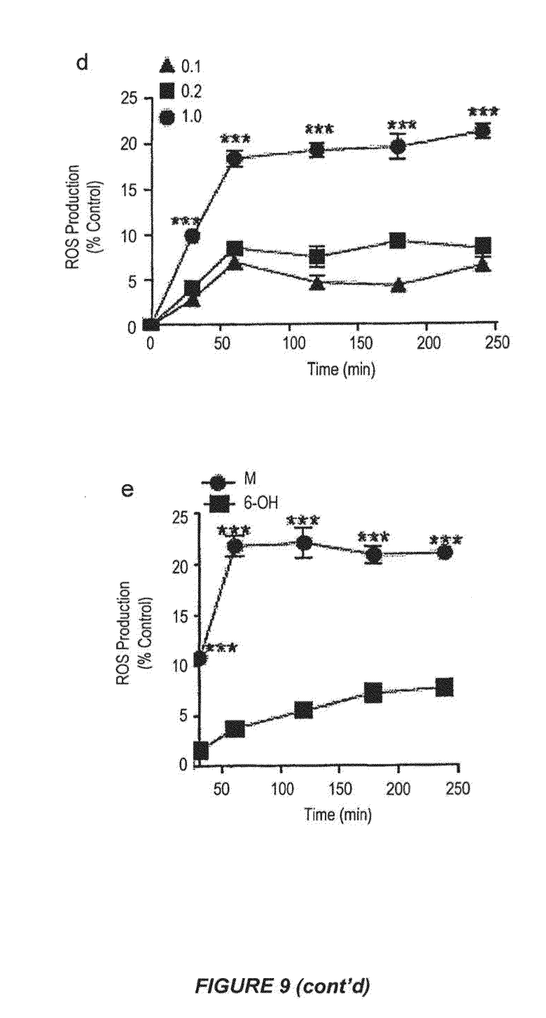

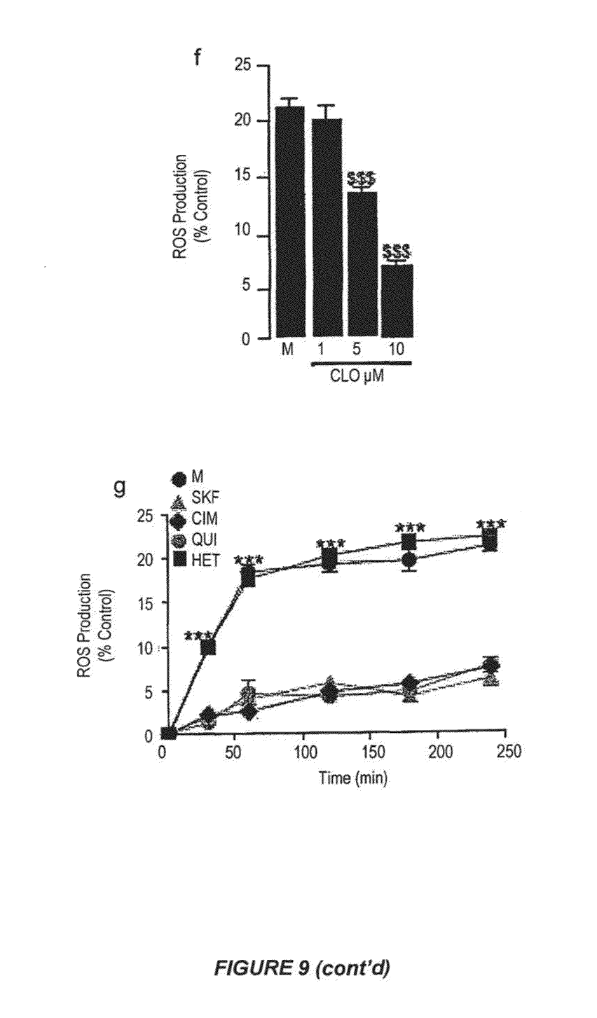

We next tested whether increased ceramide content was also leading to an accelerated aging phenotype in self-administering rats and rats treated acutely with methamphetamine. Methamphetamine self-administration resulted in elevated levels of transcription of age-related genes TNF-α, IL-6, p21, and p53 (FIG. 5a-d). Acute methamphetamine treatment resulted in increased transcription of IL-6 and p21, but not TNF-α and p53 (FIG. 5e-h). Although the cyclin-dependent kinase inhibitor p21 is usually induced by p53 dependent mechanisms, it can also be induced by p53 independent mechanisms following stress, in both cases elevated p21 transcript leads to cell cycle arrest [19]. To determine whether blockage of ceramide biosynthesis in vivo could also prevent the premature aging effects of methamphetamine we conducted an acute experiment in mice in which they were administered methamphetamine alone or methamphetamine in combination with the SPT inhibitor L-CS. We found that acute treatment with methamphetamine increased ceramide content and the expression of IL-6 and p21 mRNA (FIG. 5i-k). These methamphetamine-induced changes are as a direct result of increased ceramide content as we were able to block the increase of ceramide with the administration of L-CS and thus block the increase of IL-6 and p21 mRNA (FIG. 5 i-k). To gain more insight into the possible therapeutic use of L-CS for methamphetamine addicts we investigated whether L-CS treatment could rescue methamphetamine self-administering animals from the deleterious effects of ceramide accumulation. To test this, we allowed rats to self-administer methamphetamine for a period of 8 days, and treated them with L-CS 4 days into the self-administration study. Inhibition of de novo ceramide synthesis using L-CS blocked methamphetamine induced increases in ceramide content and transcription of age-related genes IL-6 and p21 (FIG. 5l-n). As an overall marker of the health of the animal we monitored weight, and found that L-CS treatment was able to rescue the wasting phenotype see in methamphetamine self-administering animals (FIG. 5p). However, L-CS treatment had no effect on methamphetamine self-administration (FIG. 9), nor any influence on meth induced changes in body temperature (FIG. 5o) validating that we are specifically targeting ceramide mediated effects on methamphetamine use with L-CS treatment. Together these data provide strong evidence for the possibility of treating the progeric effects of methamphetamine by inhibiting de novo ceramide biosynthesis.

### Methamphetamine Metabolism Via CYP2D6 Triggers Ceramide Biosynthesis

Methamphetamine is metabolized in humans and rodents by cytochrome P450 (CYP)-2D6, a widely distributed CYP isoform that catalyzes the oxidation of methamphetamine into D-amphetamine and 4-hydroxy-D-methamphetamine (Wu, D., et al. Biochem Pharmacol 53, 1605-1612 (1997); Lin et al. Drug Metab Dispos 25, 1059-1064 (1997)). A by-product of this reaction is the formation of reactive oxygen species (ROS) Riddle, E. L., et al., AAPS J 8, E413-418 (2006)), which are known to activate NF-KB-dependent stress-response signals that can lead to ceramide formation (Dbaibo, G. S., et al., J Biol Chem 268, 17762-17766 (1993). To determine whether CYP2D6 metabolism might be involved in methamphetamine-induced ceramide production, we blocked CYP activity in primary MEF cultures using a panel of five chemically distinct agents: clotrimazole, SKF-525A and cimetidine (three pan-CYP inhibitors), quinidine (selective for CYP2D6) and HET-0016 (selective for CYP4A). LC/MS analyses showed that incubation of MEF in the presence of clotrimazole (1-10 μM) increased the levels of non-metabolized methamphetamine (FIG. 9a) and concurrently decreased methamphetamine-induced ceramide accumulation (FIG. 9b), while exerting no effect on baseline ceramide content (in pmol-mg⁻¹ protein, control: 52.9±5; clotrimazole 10 μM: 49.2±3; n=3). Similarly to clotrimazole, SKF-525A, cimetidine and quinidine (each at 10 μM) normalized ceramide formation in D-meth-treated cells, whereas HET-0016 (10 μM) was ineffective (FIG. 9c).

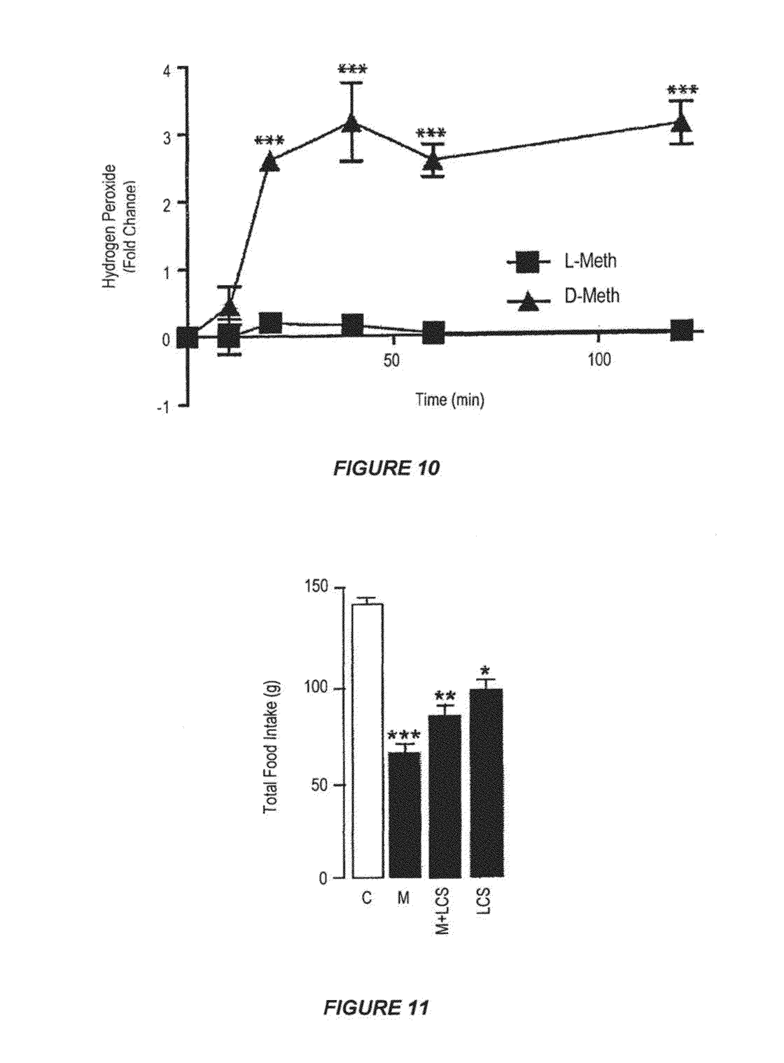

As a further test of the role of CYP2D6 in methamphetamine-induced ceramide production, we determined whether exposure to methamphetamine stimulates ROS generation in MEF. As anticipated from previous studies, methamphetamine caused a concentration-dependent increase in ROS formation (FIG. 9), whereas L-methamphetamine (a less preferred CYP2D6 substrate) and 4-hydroxy-D-methamphetamine (a product of methamphetamine metabolism via CYP2D6) had little or no effect (FIG. 9e and FIG. 10). The release of ROS evoked by methamphetamine was prevented by the pan-CYP inhibitors—clotrimazole (FIG. 9f), SKF-525A and cimetidine (FIG. 9g)—and the CYP2D6 inhibitor, quinidine (FIG. 9g), but not by the CYP4A inhibitor, HET-0016 (9g).

An early cellular response to ROS formation is the recruitment of NF-KB (Gloire, G., et al., Biochem Pharmacol 72, 1493-1505 (2006); Schreck, R., et al., EMBO J 10, 2247-2258 (1991)), which can also be induced by methamphetamine (Asanuma, M. et al. Brain Res Mol Brain Res 60, 305-309 (1998); Lee, Y. W., et al., J Neurosci Res 66, 583-591 (2001)). Accordingly, our findings indicate that the oxidative metabolism of methamphetamine via CYP2D6 stimulates ceramide biosynthesis, most likely through induction of ROS formation and subsequent engagement of NF-KB.

L-CS did not alter key centrally mediated actions of methamphetamine—including its ability to maintain self-administration (FIG. 8), increase body temperature (data not shown) and reduce food intake (FIG. 11). Nevertheless, the SPT inhibitor corrected the abnormalities in body weight.

### Discussion

Ceramide has long been implicated as a molecular modulator of aging and longevity [6]. The first evidence of this was seen with the LAG1 mutants and further supported by finding of the role of ceramide in inducing cellular senescence [10]. Our work further supports the known roles of ceramide in aging as we have found that it is not only involved in the progression of normal chronological aging, but that manipulation of its metabolism can accelerate aging. More specifically we found that alterations in de novo ceramide metabolism, caused by methamphetamine, can lead to drug-induced senescence. Although, the aging consequences of methamphetamine addiction in people were phenotypically obvious, not much was known about the molecular mechanisms responsible for this process. We have shown that methamphetamine can accelerate aging in vivo and in vitro by increasing the rate at which cells senescence and by inducing a state of chronic systemic inflammation two robust markers of aging. Of even more significance is the fact that the induction of senescence and inflammation induced peripherally by methamphetamine use is dependent on increased cellular ceramide contents and that by blocking the induction of ceramide biosynthesis with L-CS we are able to ameliorate the premature aging consequences of methamphetamine use. This may one day lead to the production of pharmacological therapies which may prolong the life of addicts in order to facilitate their recovery form methamphetamine addiction.

### Materials and Methods

### Chemicals

D-Methamphetamine hydrochloride (=amphetamine), L-methamphetamine hydrochloride, L-cycloserine and myriocin were purchased from Sigma Aldrich (St. Louis, Mo., USA). Fumonisin B₁ and C8 ceramide were purchased from Cayman Chemicals (Ann Arbor, Mich., USA). NF-κB inhibitors were purchased from Santa Cruz Biotechnology (Santa Crux, Calif., USA).

### Methamphetamine Self-Administration

Subjects.

Male Sprague-Dawley rats (Charles River, Wilmington, Mass.), weighing approximately 360-440 g at the beginning of the self-administration experiment, were individually housed in a temperature- and humidity-controlled environment under a reversed lighting 12-h light/dark cycle (lights on at 7:00 p.m.). The rats were allowed free access to food (NIH07 biscuits) in their home cage throughout the study. Water was available ad libitum in the home cage and in the testing chamber. Rats were tested in the light phase. They were experimentally and drug naïve at the beginning of this study.

Animals were maintained in facilities fully accredited by the American Association for the Accreditation of Laboratory Animals and all experiments were conducted in accordance with the guidelines of the Institutional Care and Use Committee of the Intramural Research Program, NIDA, NIH, and the Guidelines for the Care and Use of Mammals in Neuroscience and Behavioral Research (National Research Council 2003).

Apparatus.

Each of eighteen standard operant-conditioning chambers (Coulbourn Instruments, Lehigh Valley, Pa.) contained a white house light and two holes with nose-poke operanda on either side of a food trough. Upon activation, each nose poke produced a brief feedback tone. One hole was defined as active (left in nine chambers, right in remaining nine) and the other hole as inactive. methamphetamine or saline were delivered through Tygon tubing, protected by a metal spring and suspended through the ceiling of the experimental chamber from a single-channel fluid swivel. The tubing was attached to a syringe pump (Harvard Apparatus, South Natick, Mass.), which was programmed to deliver 2-s injections. The injected volume was adjusted for every animal to deliver a methamphetamine dose of 0.1 mg/kg/injection. Experimental events were controlled by microcomputers using MED Associates interfaces and software (Med Associates Inc., East Fairfield, Vt.).

Silastic catheter was implanted into the external jugular vein under anesthesia with a mixture of ketamine and xylazine (60 and 10 mg/kg i.p., respectively). Catheter exited the skin behind the ear. After catheter implantation, a nylon bolt glued to an acrylic mesh was implanted subcutaneously in the midscapular region. The nylon bolt served as a tether, preventing the catheter from being pulled out during self-administration sessions. Following surgery, the IV catheter was flushed daily during the first week with 0.2-0.3 ml of solution containing cephalosporin (100 mg/ml; Cefazolin For Injection, USP; Hospira Inc., Lake Forest, Ill., USA) and then flushed before and after each daily session with saline to maintain its patency) and then flushed after each daily session with saline to maintain its patency.

Procedure.

Each experimental group was divided into two subgroups that were tested simultaneously. One subgroup served as yoked controls and passively received an injection of saline (which was not contingent on responding) each time a response-contingent injection of 0.1 mg/kg methamphetamine was actively self-administered by the first subgroup of rats. Nose-poke responses by the yoked control rats were recorded, but had no programmed consequences. The first experimental groups consisted of 12 rats self-administering methamphetamine and 6 yoked control rats. The second experimental group consisted of 9 rats self-administering methamphetamine and 9 yoked control rats. The third experimental group consisted of 16 rats self-administering methamphetamine and 8 yoked control rats. In this third experimental group, half of the animals (8 methamphetamine and 4 yoked) received i.v. pretreatment with L-cycloserine (L-CS) that started before session 4. The pretreatment with L-CS was always given immediately before and after each session and the catheter was flushed with 0.5 ml of saline afterwards. Before and after sessions 4 and 5 animals received dose 10 mg/kg of L-CS and before and after sessions 6, 7, and 8 they received dose 20 mg/kg of L-CS. The animals received total dose 160 mg/kg of L-CS during the experiment. In this group, we also measured three times the rectal temperature. The first measurement was done one the day before the experiment began at 2 pm, the second measurement was done at 2 pm before the eighth session, and the third measurement immediately after the eighth session.

Eight consecutive 15-hour sessions were conducted between 4 p.m. and 8 a.m. Rats and the food in the feeders were weighed before the start of each session. At the start of each session, a white house light was turned on and a priming injection of 0.1 mg/kg methamphetamine (or saline for yoked group), sufficient to fill the “dead” space of the IV catheter, was automatically delivered. Rats learned to self-administer methamphetamine under one-response, fixed ratio schedule (FRI) of i.v. methamphetamine injection with 30-s time-out duration. Each nose-poke response in the active hole (FRI) produced a delivery of an i.v. injection of 0.1 mg/kg of methamphetamine followed by 30-s timeout period, during which the chamber was dark and responses in either hole had no programmed consequences. Nose-poke responses in the “inactive” hole were recorded but had no programmed consequences.

Tissue Collection.

The rats were always euthanized 2 hrs after the eighth session ended. The first experimental group of rats was euthanized by decapitation and brain, liver, heart, kidney (left), spleen, pancreas, testis, epididymal fat, skeletal muscle (vastus lateralis muscle), and skin (hind paw) were harvested from each rat. All tissues were rinsed in the mix of RNase-free water with DEPC-treated phosphate-buffered saline (PBS) and dried with sterile gauze. Brains and livers were snap-frozen in isopentane. The rest of the tissues were snap-frozen in liquid nitrogen. All tissues were double wrapped in aluminum foil and stored in −80° C.

The second experimental group was euthanized as follows. Five yoked pairs were euthanized by decapitation and tissues were harvested and handled as described above. Four yoked pairs were anaesthetized with Equithesin (9.72 mg/ml pentobarbital and 44.4 mg/ml chloral hydrate, 3 ml/kg i.p.) and perfused intracardially with 0.1M PBS followed by 4% paraformaldehyde dissolved in 0.1M PBS. Animals were then decapitated and brains removed and fixed in 4% paraformaldehyde in 0.1M PBS for 2 h and then immersed in 20% sucrose/0.1 M PBS solution for 48 h at 4° C. The brains were subsequently rapidly frozen in dry ice and stored at −80° C.

The third experimental group was euthanized by decapitation (see the first group for details) and brain, liver, heart, spleen, left kidney, skeletal muscle and skin were collected from each rat. The tissues were handled as described for the first group.

Drugs.

S(+)-methylamphetamine HCl (methamphetamine) was purchased from Sigma Aldrich (St. Louis, Mo., USA) and dissolved in saline. L-cycloserine (Sigma Aldrich, St. Louis, Mo., USA) was dissolved in saline and administered i.v. in volume 1 ml/kg.

### Acute Methamphetamine Administration in Rats

Subjects.

Forty-six male Sprague-Dawley rats (Charles River, Wilmington, Mass.), weighing approximately 360-420 g were used in these experiments. Other details as described in methamphetamine self-administration section.

Procedure.

Three groups of rats were used for these experiments. Group 1: On the day of the experiment, rats received two i.p. injections of methamphetamine, 10 mg/kg (n=6), or saline (n=6) every 2 h. Two hours after the last administration of methamphetamine or saline, rats were euthanized by decapitation. Brain, liver, kidney (left), heart, skin (hind paw), skeletal muscle (vastus lateralis muscle) were harvested. Brains and livers were snap-frozen in isopentane and other tissues were snap-frozen in liquid nitrogen and stored at −80° C. Group 2: On the day of the experiment, rats received two i.p. injections of methamphetamine, 1.5 mg/kg (n=6), or saline (n=6) every 2 h. Other details are the same as for group 1. Group 3: On the day of the experiment, five rats received two i.p. injections of 1.5 mg/kg methamphetamine every 2 h; five rats received two i.p. injections of 5 mg/kg methamphetamine every 2 h; six rats received two i.p. injections of 10 mg/kg D-methamphetamine every 2 h, and six rats received two i.p. injections of saline every 2 h. Two hours after the last injection, blood was collected by cardiac puncture under isoflurane anesthesia and immediately afterwards brain, skeletal muscle and skin were harvested and handled as described for group 1.

Drugs.

S(+)-methylamphetamine HCl (methamphetamine) was purchased from Sigma Aldrich (St. Louis, Mo., USA) and dissolved in saline.

Lipid Extractions from Tissues

Lipid extractions were conducted as previously described [20]. Briefly, frozen brain samples were weighed and homogenized in cold methanol containing appropriate internal standards (listed below). Lipids were extracted by adding chloroform and water (2/1, vol/vol) and fractionated through open-bed silica gel columns by progressive elution with chloroform/methanol mixtures. Fractions eluted from the columns were dried under nitrogen, reconstituted in chloroform/methanol (1:4, vol/vol; 0.1 ml) and subjected to LC/MS.

Lipid Extractions from Cells in Cultures

Cells were washed with ice-cold phosphate-buffered saline (PBS) and scraped into 0.5 ml of methanol/water (1:1, vol:vol) containing the internal standards listed below. Protein concentrations were measured using the BCA protein assay (Pierce, Rockford, Ill., USA). Lipids were extracted with chloroform/methanol (2:1, vol:vol; 1.0 mL). The organic phases were collected, dried under nitrogen and dissolved in methanol for LC/MS analyses.

### Lipidomic Analyses.

Lipid molecular species were quantified by normalizing the individual molecular ion peak intensity with an internal standard for each lipid class. A mixture of non-endogenous molecules was used as internal standards and added before the extraction process to allow lipid levels to be normalized for both extraction efficiency and instrument response.

Fatty Acids.

Fatty acids were quantified with an Agilent 1100 liquid chromatograph coupled to a 1946D mass detector equipped with an ESI interface (Agilent Technologies, Palo Alto, Calif.). A reversed-phase XDB Eclipse C18 column (50×4.6 mm i.d., 1.8 μm, Zorbax, Agilent Technologies) was eluted with a linear gradient from 90% to 100% of A in B for 2.5 min at a flow rate of 1.5 ml/min with column temperature at 40° C. Mobile phase A consisted of methanol containing 0.25% acetic acid and 5 mM ammonium acetate; mobile phase B consisted of water containing 0.25% acetic acid and 5 mM ammonium acetate. Column temperature was kept at 40° C. Mass detection was in the negative ionization mode, capillary voltage was set at −4.0 kV and fragmentor voltage was 120 V. Nitrogen was used as drying gas at a flow rate of 13 liters/min and a temperature of 350° C. Nebulizer pressure was set at 60 pounds per square inch. For quantification purposes, the deprotonated pseudo-molecular ions [M−H]⁻ of the fatty acids were monitored in the selected ion-monitoring mode (SIM), using d₈-arachidonic acid (Cayman Chemical, Ann Arbor, Mich.) as internal standard (m/z=311.3) as previously reported (ref) Commercially available fatty acids (Nu-Chek Prep, Elysian, Minn., Cayman Chemical or Sigma-Aldrich, St Louis, Mo.) were used as references.

Monoacylglycerols (AGs).

We used an Agilent 1100-LC system (Agilent Technologies, Palo Alto, Calif.) coupled to a 1946D-MS detector equipped with an ESI interface (Agilent Technologies). MGs were separated on a XDB Eclipse C18 column (50×4.6 mm i.d., 1.8 ρm; Zorbax; Agilent Technologies). They were eluted with a gradient of methanol in water (from 85% to 90% methanol in 2.0 min and 90% to 100% in 3.0 min) at a flow rate of 1.5 ml/min. Column temperature was kept at 40° C. MS detection was in the positive ionization mode, capillary voltage was set at 3 kV, and fragmentor voltage was 120 V. Nitrogen was used as drying gas at a flow rate of 13 liters/min and a temperature of 350° C. Nebulizer pressure was set at 60 psi. Commercial MGs were used as reference standards. For quantification purposes, we monitored the Na+ adducts of the molecular ions [M+Na]+ in SIM mode, using HDG (m/z 367) as an internal standard.

Diacylglycerols (DGs).

We used an Agilent 1100-LC system coupled to a MS detector Ion-Trap XCT interfaced with ESI (Agilent Technologies). DG species were separated using a XDB Eclipse C18 column (50×4.6 mm i.d., 1.8 μm, Zorbax), eluted by a gradient of methanol in water (from 85% to 90% methanol in 2.5 min) at a flow rate of 1.5 ml/min. Column temperature was kept at 40° C. The capillary voltage was set at 4.0 kV and skimmer voltage at 40 V. N_{itrogen} was used as drying gas at a flow rate of 12 liters/min, temperature at 350° C., and nebulizer pressure at 80 psi. Helium was used as collision gas, and fragmentation amplitude was set at 1.2 V. DG were identified in the positive ionization mode based on their retention times and MS³ properties, using synthetic standards as references. Multiple reaction monitoring was used to acquire full-scan tandem MS spectra of selected DG ions. Extracted ion chromatograms were used to quantify isobaric DG species and dinonadecadienoin (m/z 667.8>367.5), which was used as an internal standard.

Triacylglycerols (TGs).

We used an Agilent 1100-LC system coupled to a MS detector Ion-Trap XCT interfaced with atmospheric pressure chemical ionization (Agilent Technologies). Lipids were separated on a Poroshell 300SB C18 column (2.1×75 mm i.d., 5 μm, Agilent Technologies) at 50° C. A linear gradient of methanol in water containing 5 mM ammonium acetate and 0.25% acetic acid (from 85% to 100% of methanol in 4 min) was applied at a flow rate of 1 ml/min. MS detection was set in positive mode. Corona discharge needle voltage set at 4 kV. Capillary voltage was 4.0 kV, skim1 40 V, and capillary exit at 118 V. Nitrogen was used as drying gas at a flow rate of 10 liters/min, temperature of 350° C., nebulizer pressure of 50 PSI and vaporization temperature at 400° C. Helium was used as collision gas. Total TGs were quantified by integrating the area of the total ion current (m/z 700-900) at a selected interval of retention time (from 4 to 5 min), using TG 19:1/19:1/19:1 (m/z 944.8, Nu-Chek Prep) as an internal standard.

Glycerophospholipids.

Phospholipids molecular species were analyzed by tandem mass spectrometry, using an Agilent 1100 liquid chromatograph coupled to an ESI-ion-trap XCT mass detector. A reversed-phase Poroshell 300SB C18 column (2.1×75 mm i.d., 5 μm, Agilent) was eluted with a linear gradient from 85% to 100% of mobile phase A in B in 5 min at a flow rate of 1.0 ml/min with column temperature at 50° C. Mobile phase composition was as described above. The capillary voltage was set at 4.0 kV and skimmer voltage at −40 V. Nitrogen was utilized as drying gas at a flow rate of 10 liters/min, temperature at 350° C. and nebulizer pressure at 60 pounds per square inch. Helium was the collision gas and fragmentation amplitude was set at 1.2 V. Mass detection was in the negative ionization mode and was controlled by the Agilent/Bruker Daltonics software version 5.2. Synthetic 1,2-diheptadecanoyl-sn-glycero-3-phosphoethanolamine, 1,2-diheptadecanoyl-sn-glycero-3-phosphocholine, 1,2-diheptadecanoyl-sn-glycero-3-phosphoglycerol, 1,2-diheptadecanoyl-sn-glycero-3-phosphoserine, 1,2-diheptadecanoyl-sn-glycero-3-phosphoinositol (Avanti Polar Lipids, Alabaster, Ala.) were used as internal standards.

Sphingolipids.

Sphingolipid molecular species were analyzed by tandem mass spectrometry, using an Agilent 1100 liquid chromatography coupled to an ESI-ion-trap XCT mass detector. Dihydroceramides and ceramides were separated on a Poroshell 300 SB C18 column (2.1×75 mm i.d., 5 μm; Agilent Technologies) maintained at 30° C. A linear gradient of methanol in water containing 5 mM ammonium acetate and 0.25% acetic acid (from 80% to 100% of methanol in 3 min) was applied at a flow rate of 1 ml/min. Detection was in the positive mode, capillary voltage was 4.5 kV, skim1 −40 V, and capillary exit −151 V. Nitrogen was used as drying gas at a flow rate of 12 L/min, temperature of 350° C., and nebulizer pressure of 80 psi. Helium was used as collision gas. Ceramide species were identified by comparison of its LC retention time and MSⁿ fragmentation pattern with that of authentic standards (Avanti Polar Lipids). Extracted ion chromatograms were used to quantify the following ceramides: d18:1/16:0 [M+H]⁺ (m/z=538.5>520.5>264.3), d18:0/16:0 [M+H]⁺ (m/z=540.5>522.5), d18:1/18:0 [M+H]⁺ (m/z=566.5>548.5>264.3), d18:0/18:0 [M+H]⁺ (m/z=568.5>550.5), d18:1/24:0 [M+H]⁺ (m/z=650.6>632.8>2643), d18:0/24:0 [M+H]⁺ (m/z=652.6>634.8), d18:1/24:1 [M+H]⁺ (m/z=648.6>630.8>264.3), d18:0/24:1 [M+H]⁺ (m/z=650.6>632.8), using d18:1/12:0 [M+H]⁺ (m/z=482.5>464.5>264.3) as an internal standard. Sphingomyelin species were separated using a reversed-phase Poroshell 300SB C18 column (2.1×75 mm i.d., 5 μm, Agilent) and eluted with a linear gradient from 85% to 100% of mobile phase A in B in 5 min at a flow rate of 1.0 ml/min with column temperature at 50° C. Mobile phase composition was as described above. The capillary voltage was set at −4.0 kV and skimmer voltage at −40 V. Nitrogen was utilized as drying gas at a flow rate of 10 liters/min, temperature at 350° C. and nebulizer pressure at 60 pounds per square inch. Helium was the collision gas and fragmentation amplitude was set at 1.2 V. Mass detection was set in either positive or negative ionization mode and was controlled by the Agilent/Bruker Daltonics software version 5.2. Sphingomyelin species were identified by LC-MSⁿ using reference standards (Avanti Polar Lipids) and quantified using sphingomyelin d18:1/12:0 [M]+(m/z=647.8>588.8) as an internal standard. Sphingomyelin species were monitored using the following multiple-ion reactions: d18:1/16:0 [M]⁺ (m/z=703.8>644.8), d18:1/18:0 [M]⁺ (m/z=731.8>672.8), d18:1/24:0 [M]⁺ (m/z=815.8>756.8), d18:1/24:1 [M]⁺ (m/z=813.8>754.8).

### Gene Expression.

Total RNA was extracted from frozen tissues using TRIzol reagent (Invitrogen, Carlsbad, Calif.) and was purified with the RNeasy mini kit (Qiagen, Valencia, Calif.). First-strand complementary DNAs were synthesized using SuperScript II RNaseH reverse transcriptase (Invitrogen). Reverse transcription of total RNA (2 μg) was carried out using oligo(dT)12-18 primers for 50 min at 42° C. mRNA levels were measured by quantitative real-time polymerase chain reaction (RT-PCR) with a Mx 3000P system (Stratagene, La Jolla, Calif.). The following list of primers and fluorogenic probes purchased from Applied Biosystems (TaqMan Gene Expression Assays, Foster City, Calif.): Ceramide Synthase 1 (Rn01420081_m1), Ceramide Synthase 2 (Rn01762789_m1), Ceramide Synthase 4 (Rn01767402_m1), Ceramide Synthase 5 (Rn01532864_m1), Ceramide Synthase 6 (Rn01270930_m1), Interleukin-6 (Rn01410330_m1). mRNA levels were normalized using beta actin, 18S ribosomal protein or glyceraldehyde-3-phosphate dehydrogenase as internal standards. Additional PCR primers were designed using Primer 3 (https://www.Frodo.wit.mit.edu), and the sequences are shown in S.Table XXX.

### Enzymatic Activities

Fresh rat tissues were collected in 1 ml of homogenization buffer (25 mM HEPES, pH 7.4, containing 5 mM EGTA, 50 mM NaF, and complete mini EDTA-free protease inhibitor). Tissues were disrupted using pulse homogenizer, and centrifuged at 800×g for 5 min. The postnuclear supernatant was centrifuged at 250,000×g for 30 min at 4° C. in ultracentrifuge. The microsomal membrane pellet was resuspended in 250-500 μl of homogenization buffer. Protein concentration was measured using the BCA protein assay (Pierce, Rockford, Ill.).

### (Dibydro)Ceramide Synthase Activity

Ceramide synthase activity was measured at 37° C. for 1 hr in HEPES buffer (20 mM, pH 7.4) containing 2 mM MgCl₂, fatty acid-free bovine serum albumin (20 μmol) membrane proteins (0.05-0.1 mg), using dihydrosphingosine (sphinganine d17:0, 20 μmol) and palmitoyl-coenzyme A (70 μmol) as substrates. The reactions were stopped by adding chloroform-methanol (2:1, v/v) containing C12:0 ceramide (d18:1/12:0) as an internal standard. Lipid extracts were dried under nitrogen and reconstituted in chloroform-methanol (1:3, v/v; 0.1 ml) for LC-MS analyses. Products of reaction were measured using an Agilent 1100-LC system coupled to ion-trap XCT and interfaced with ESI (Agilent Technologies). The mobile phase A was methanol containing 0.25% acetic acid and 5 mM ammonium acetate; mobile phase B was water containing 0.25% acetic acid and 5 mM ammonium acetate. Lipids were separated using a reversed-phase Poroshell 300SB C-18 column (2.1×75 mm i.d., coating layer of 0.25 μm on total particle diameter of 5 μm, 300 Å of porous diameter, Agilent Technologies) and identified based on their retention times. A linear gradient was applied from 50% A to 100% B in 6 min at a flow rate of 1.0 ml/min with column temperature set at 50° C. The capillary voltage was set at 4.5 kV and skimmer voltage at 40V. Nitrogen was used as drying gas at a flow rate of 10 liters/min, temperature at 350° C. and nebulizer pressure at 60 psi. Helium was used as collision gas. For quantification purposes, we monitored the ions at m/z 526.5>508.5 for C17:0 dihydroceramide (d17:0/16:0) and m/z 482.5>464.5>264.3 for C12:0 ceramide (d18:1/12:0).

### Primary Fibroblast Culture

Mouse embryonic fibroblasts (MEFs) were prepared from mice embryo as previously reported [21]. Briefly, pregnant mice at day 13 post coitum were sacrificed and the uteri were dissected out. Each embryo was separated from the placenta and the head and visceral tissues were removed. The remaining body was minced in PBS and incubated with 0.1 mM trypsin/1 mM EDTA at 37° C. for 15 min. Two volumes of Dulbecco's Modified Eagle Medium (DMEM) containing 10% fetal bovine serum (FBS) was added and stood for 5 minutes to settle down large pieces of unbroken tissues. The supernatant was then removed and centrifuged at 200×g for 5 min and suspended in fresh DMEM containing 10% FBS. Cells were cultured at 37° C. with 5% CO_{2.}

### Mitochondrial Isolation

### Senescence-Associated β-Galactosidase (β-Gal) Staining.

Detection of senescence-associated β-galactosidase staining was performed as previously reported [22]. Briefly, we plated MEFs on Lab-Tek chamber slides at a density of 5×10⁴ cells per chamber. Next day, cells were treated with the indicated dose of drug for 48 hours. Cells were washed twice with PBS and fixed in 2% formaldehyde/0.2% glutaraldehyde for five minutes at room temperature. After two PBS washes the slides were incubated with fresh β-galactosidase stain solution (1 mg/mL 5-bromo-4-chloro-3-indoyl β-D-galactoside (X-Gal) 40 mM sodium phosphate, pH 6.0, 5 mM potassium ferrocyanide, 5 mM potassium ferricyanide, 150 mM NaCl, and 2 mM MgCl₂) at 37° C. for 12-16 hours. Slides were washed with PBS and mounted with DAPI containing media. Images were taken for the β-gal staining and overlapped with fluorescent images taken of the same region to count the number of cells staining positive for β-gal (Nikon Eclipse E600). More than 200 cells from 5 different regions of a slide were counted in a blind fashion.

### DNA Replication Assay

Cells were seeded in 12-well plates (5×10⁴ per well) and treated with 1 mM methamphetamine for 48 hours. The medium was then replaced with fresh media containing 2.5 μCi/mL of [³H]thymidine (6.7 Ci/mmol, MPBio). After 24 hours, cells were rinsed twice with ice-cold PBS and genomic DNA was isolated using the DNeasy Kit (Qiagen). Radioactivity from the genomic DNA was measured in a liquid scintillation counter LS-6500 (Beckman Instruments, Fullerton, Calif.).

### Crystal Violet Staining

To study cell morphology, cells were plated and treated in 6-well plates. After fixation, with 4% paraformaldehyde cells were stained with crystal violet (0.5% crystal violet in methanol:PBS, 1:1, v/v). After thorough wash in tap water, images were taken using a Westover Scientific Series 8 microscope.

### Population Doubling

We plated 1×10⁶ cells on a 60 mm dishes and treated them with 1 mM methamphetamine. Cells were trypsinized and re-plated at the same density (1×10⁶ cells/dish) every 3 days for 6 passages. Population doublings were calculated according to the formula log (final cell number/plated cell number/log 2).

### Statistical Analyses

Lipid-level data were analyzed using restricted maximum likelihood estimation (Proc Mixed; SAS Institute, Cary, N.C.), which can handle data sets in which some points are missing “at random” (e.g., due to technical problems) and which does not require homoscedasticity between conditions. Methamphetamine exposure was a between-subjects factor, and lipid species (or lipid family) was a within-subjects factor. Residuals under this mixed model were found to be normally distributed. P values from Proc Mixed were used to perform paired comparisons between the methamphetamine and control group for each lipid species (or family), maintaining an overall false discovery rate (Benjamini and Hochberg, 1995) of 0.05 for the entire experiment. For graphic presentation of group results, heatmaps were generated using the Studentized value for each comparison, such that each cell represents the size of the difference between the means of the methamphetamine and control groups, divided by the pooled standard error. Red cells indicate increased lipid levels in the methamphetamine group, and green cells represent decreased levels. For graphic presentation of individual-subject results, heatmaps were generated by normalizing the data for each lipid species relative to the mean and standard error of the control group, such that the color of each subject's cell indicates the number of standard errors above (red cells) or below (green cells) the mean of the control group. Descriptive statistics are presented as means±SD. The differences between unadjusted mean values were determined by two-tailed t-test. All confidence intervals correspond to a 95% confidence level.

U.S. Provisional Patent Application Ser. No. 61/806,335, filed on Mar. 28, 2013, is incorporated herein by reference in its entirety and particularly with respect to its updated and more comprehensively described research methods and results and discussion of their therapeutic utility.

### ROS Production

Production of ROS was measured using the fluorescent probe CM-H2DCFDA (Invitrogen). This carboxy derivative of fluorescein carries additional negative charges that improve its retention compared to noncarboxylated forms. For these experiments, MEFs were grown in DMEM without phenol red, for which anti-oxidative properties have been reported. Cells were plated to subconfluence in 12-well plates, washed 3 times with pre-warmed PBS and loaded for 30 min at 37° C./5% CO₂ with 5 mM CM-H2DCFDA in DMEM without phenol red (loading medium). Then the loading medium was removed and pre-warmed fresh medium containing the different CYP450 inhibitors in presence or absence of methamphetamine was added. Fluorescence (excitation at 485 nm, emission at 530 nm) was analyzed immediately, cells were incubated at 37° C./5% CO₂, and fluorescence was analyzed at the indicated time points. ROS rate versus control (%) was calculated subtracting the percentage of ROS increasing from time zero in the methamphetamine-treated samples to the percentage of ROS increasing from time zero in vehicle-treated samples.

### REFERENCES

1. Maeno, Y., et al., Direct effects of methamphetamine on hypertrophy and microtubules in cultured adult rat ventricular myocytes. Forensic Sci Int, 2000. 113(1-3): p. 239-43.
2. Lau, J. W., S. Senok, and A. Stadlin, Methamphetamine-induced oxidative stress in cultured mouse astrocytes. Ann N Y Acad Sci, 2000. 914: p. 146-56.
3. Wu, C. W., et al., Enhanced oxidative stress and aberrant mitochondrial biogenesis in human neuroblastoma SH-SY5Y cells during methamphetamine induced apoptosis. Toxicol Appl Pharmacol, 2007. 220(3): p. 243-51.
4. Karch, S. B., B. G. Stephens, and C. H. Ho, Methamphetamine-related deaths in San Francisco: demographic, pathologic, and toxicologic profiles. J Forensic Sci, 1999. 44(2): p. 359-68.
5. D'Mello N, P., et al., Cloning and characterization of LAG1, a longevity-assurance gene in yeast. J Biol Chem, 1994. 269(22): p. 15451-9.
6. Jazwinski, S. M. and A. Conzelmann, LAG1 puts the focus on ceramide signaling. Int J Biochem Cell Biol, 2002. 34(11): p. 1491-5.
7. Laviad, E. L., et al., Characterization of ceramide synthase 2: tissue distribution, substrate specificity, and inhibition by sphingosine 1-phosphate. J Biol Chem, 2008. 283(9): p. 5677-84.
8. Pewzner-Jung, Y., S. Ben-Dor, and A. H. Futerman, When do Lasses (longevity assurance genes) become CerS (ceramide synthases)?: Insights into the regulation of ceramide synthesis. J Biol Chem, 2006. 281(35): p. 25001-5.
9. Modrak, D. E., et al., Ceramide regulates gemcitabine-induced senescence and apoptosis in human pancreatic cancer cell lines. Mol Cancer Res, 2009. 7(6): p. 890-6.
10. Miller, C. J. and G. H. Stein, Human diploid fibroblasts that undergo a senescent-like differentiation have elevated ceramide and diacylglycerol. J Gerontol A Biol Sci Med Sci, 2001. 56(1): p. B8-19.
11. Koob, G. and M. J. Kreek, Stress, dysregulation of drug reward pathways, and the transition to drug dependence. Am J Psychiatry, 2007. 164(8): p. 1149-59.
12. Levy, M. and A. H. Futerman, Mammalian ceramide synthases. IUBMB Life, 2010. 62(5): p. 347-56.
13. Campisi, J., From cells to organisms: can we learn about aging from cells in culture? Exp Gerontol, 2001. 36(4-6): p. 607-18.
14. Hwang, E. S., G. Yoon, and H. T. Kang, A comparative analysis of the cell biology of senescence and aging. Cell Mol Life Sci, 2009. 66(15): p. 2503-24.
15. Schneider, E. L. and Y. Mitsui, The relationship between in vitro cellular aging and in vivo human age. Proc Natl Acad Sci USA, 1976. 73(10): p. 3584-8.
16. Chen, X., et al., Senescence-like changes induced by expression of p21(waf1/Cip1) in NIH3T3 cell line. Cell Res, 2002. 12(3-4): p. 229-33.
17. Erickson, S., et al., Involvement of the Ink4 proteins p16 and p15 in T-lymphocyte senescence. Oncogene, 1998. 17(5): p. 595-602.
18. Johnson, T. E., Recent results: biomarkers of aging. Exp Gerontol, 2006. 41(12): p. 1243-6.
19. Gartel, A. L. and A. L. Tyner, The role of the cyclin-dependent kinase inhibitor p21 in apoptosis. Mol Cancer Ther, 2002. 1(8): p. 639-49.
20. Astarita, G., F. Ahmed, and D. Piomelli, Lipidomic analysis of biological samples by liquid chromatography coupled to mass spectrometry. Methods Mol Biol, 2009. 579: p. 201-19.
21. Conner, D. A., Mouse embryo fibroblast (MEF) feeder cell preparation. Curr Protoc Mol Biol, 2001. Chapter 23: p. Unit 23 2.
22. Kurz, D. J., et al., Senescence-associated (beta)-galactosidase reflects an increase in lysosomal mass during replicative ageing of human endothelial cells. J Cell Sci, 2000. 113 (Pt 20): p. 3613-22.

Unless otherwise stated, the following terms used in the specification and claims are defined for the purposes of this Application and have the following meanings. It is noted here that as used in this specification and the appended claims, the singular forms “a,” “an,” and “the” include plural reference unless the context clearly dictates otherwise.

Each publication, patent application, patent, and other reference cited in any part of the specification is incorporated by reference in its entirety. With regard to any inconsistencies in usage, the present disclosure will dominate. Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

1. A method of treating an amphetamine-type drug-induced toxicity, said method comprising administering to the subject in need thereof an effective amount of a modulator of ceramide metabolism or apoptosis which counters an effect of the amphetamine-type drug on ceramide levels or ceramide metabolism or apoptosis.

2. The method of claim 1, wherein the treating reduces, prevents or delays the development of an amphetamine-type drug toxicity selected from induced senescence or organ failure in the subject.

3. The method of claim 1, wherein the toxicity is mediated by an amphetamine-type drug-induced increased ceramide signaling in apoptosis.

4. The method of claim 3, wherein the amphetamine-type drug is administered to the subject before or after the modulator.

5. (canceled)

6. The method according to claim 4, wherein the modulator is administered from about 15 minutes to about 24 hours before administering the amphetamine-type drug.

7. (canceled)

8. The method according to claim 3, wherein the modulator is administered at substantially the same time the amphetamine-type drug.

9. The method according to claim 4, wherein the modulator is administered from about 15 minutes to about 24 hours after administering the amphetamine-type drug.

10. The method of claim 1 wherein the modulator is a ceramide synthesis inhibitor.

11. The method of claim 10, wherein the modulator which inhibits ceramide biosynthesis is an antisense nucleic acid, a ribozyme, a triplex-forming oligonucleotide, a siRNA, a probe, a primer, an antibody or a combination thereof.

12. The method of claim 11, wherein the modulator targets at least one ceramide-biosynthetic enzyme selected from the group consisting of a sphingomyelinase, serine palmitoyltransferase, 3-ketosphinganine reductase, ceramide synthase, dihydroceramide desaturase, and combinations thereof.

13. The method of claim 12, wherein the modulator binds to the targeted enzyme to inhibit the enzyme or binds to a nucleic acid encoding the enzyme to reduce the expression of the protein.

14. The method according to claim 12, wherein the modulator comprises a compound selected from the group consisting of FB1, D609, myriocin, cycloserine, thalidomide, lenalidomide and combinations thereof.

15. The method according to claim 1, wherein the modulator is adalimumab, golimumab, infliximab, natalizumab, etanercept, Certolizumab pegol, and Pegsunercept.

16. The method of claim 1, wherein the toxicity is atherosclerosis, cardiomyopathy, cardiac infarction, cardiac insufficiency, or a decline in kidney function.

17. The method of claim 1 wherein the subject is human.

18. The method of claim 1 wherein the amphetamine-type drug is selected from the group consisting of amphetamine, dextroamphetamine, ephedrine, pseudoephedrine, methamphetamine, methylphenidate and the pharmaceutically acceptable salts thereof.

19. The method of claim 1, wherein the modulator is cycloserin.

20. A pharmaceutical composition comprising a therapeutically effective amount of an amphetamine-type drug; and a therapeutically effective amount of an agent that inhibits ceramide biosynthesis or apoptosis; and a pharmaceutically acceptable carrier.

21. A pharmaceutical composition according to claim 20, wherein the agent that inhibits ceramide biosynthesis targets at least one ceramide-biosynthetic enzymes selected from the group consisting of a sphingomyelinase, serine palmitoyltransferase, 3-ketosphinganine reductase, ceramide synthase, dihydroceramide desaturase, and combinations thereof.

22. A pharmaceutical composition according to claim 20, wherein the agent that inhibits ceramide biosynthesis comprises a compound selected from the group consisting of Fumonisin B1 (FB1), tyclodecan-9-xanthogenate (D609), myriocin and combinations thereof.
